(19) United States
(12) Patent Application Publication
(10) Pub. No.: US 2006/0173409 A1
(43) Pub. Date: Aug. 3, 2006

(54) Safety syring

(76) Inventor: Chang-Ming Yang, Miaoli (TW)

Correspondence Address: HDSL, 4331 STEVENS BATTLE LANE, FAIRFAX, VA, 22033, US

(21) Appl. No.: 11/022,677

(22) Filed: Dec. 28, 2004

(51) Int. Cl. A61M 5/00; A61M 5/32

(52) U.S. Cl. 604/110; 604/192

(57) ABSTRACT

A safety syringe, particularly, referring to a safety structure formed as a part in the syringe barrel mainly includes: a barrel with a rubber stopper in its interior cavity, where a shallow disk indention is at the rear surface of the rubber stopper, an annular check rib in the rear portion of inner side of the barrel to restrain the stopper from pulling out of the barrel, a needle body with the fused needle engaging with the front portion of the barrel, and an actuator, which involves an electromagnet and a magnet prior to the electromagnet with distinct magnetic poles between them and results in the induced magnetic force for the purpose of the reciprocal movement in the interior cavity of the barrel by way of engaging the magnet on the front edge of the actuator with the disk indention in the distal surface of the rubber stopper and switching the electromagnetic pole.

### BACKGROUND OF THE INVENTION

1. Field of the Invention

The present invention relates to a safe syringe, more particularly, to a single use safety syringe, and means in which, before or after the manipulation of injecting or aspirating, healthcare professionals can operate in the rear of the needle cannula tip to avoid the risk of accidental needlestick.

2. Description of the Prior Art

In general, a conventional syringe set includes a syringe and a needle cap on it; the needle cannula is fused into needle body at the front end of the syringe. A needle cap is truncated-conical in shape with an aperture at the proximal end and a cavity/housing inside the needle cap.

In the conventional syringe operation, the operator needs to remove the needle cap from the syringe and insert the needle cannula into a patient's the injection site. However, after the procedure of injecting or aspirating, the reposition of the cap may cause the needlestick injuries to healthcare professionals, which provide ways of virus infection and biomedical contamination.

Thereby, the modifications of these foregoing drawbacks of the conventional syringes are urgently demanded.

In accordance with the every shortcoming described above, the inventor, as a M.A., devotes his efforts on the R&D of safety syringe and succeeds to create these novel inventions of safety syringe.

### SUMMARY OF THE INVENTION

It is thus an object of this invention to offer novel structures of safe syringe. It is also a further object to avoid the reuse of syringe and provide a self-destruct mechanism after the completion of injecting or aspirating. It is still a further object to protect healthcare workers from accidental needle puncture and prick.

Those objectives are accomplished by the present inventions of safety syringe having:

a barrel with a rubber stopper inside, which have a groove and a annular check rib formed at the rear end of the (i.e. a structure to stop this rubber stopper leaving from the barrel.) near the rear portion of the interior of this barrel, and a needle hub/holder which has the fused needle;

an actuator having an electromagnet and a magnet prior to it with an unlike pole;

means that the actuator is plugged into the barrel from the rear side;

a magnet engaging the groove at the rear end of the plug and in front of the forgoing actuator; and
means of switching the pole of the electromagnet, which cause the plug moved reciprocally inside said barrel due to the attraction and repellence between said electromagnet and said permanent magnet.

### BRIEF DESCRIPTION OF THE DRAWINGS

The drawings disclose an illustrative embodiment of the present invention which serves to exemplify the various advantages and objects hereof, and are as follows:

FIG. 1 of the drawing shows a sectional assembled view of safety syringe of the present invention;

FIG. 2 of the drawing is a sectional view of safety syringe which illustrates the motion of its mechanism;

FIG. 3 of the drawing is a sectional view of safety syringe which illustrates the subsequent motion of its mechanism;

FIG. 4 of the drawing is a sectional view of safety syringe of the invention according to the first embodiment;

FIG. 5 of the drawing is a sectional view of safety syringe of the invention according to the second embodiment;

FIG. 5A of the drawing is a sectional view of safety syringe of the invention according to the third embodiment;

FIG. 6 of the drawing is a sectional view of safety syringe of the invention according to the fourth embodiment;

FIG. 7 of the drawing is an elevational view of safety syringe of the invention according to the fifth embodiment;

FIG. 8 of the drawing is a sectional view of safety syringe of the invention according to the fifth embodiment;

FIG. 9, FIG. 10, FIG. 11, FIG. 12, FIG. 13, FIG. 14, and FIG. 15 of the drawings are sectional views of safety syringe of the invention according to the fifth embodiment illustrating the sequence of operation of the syringe;

FIG. 16 of the drawing is a sectional view of safety syringe of the invention according to the sixth embodiment;

FIG. 17 of the drawing is an elevational view of safety syringe of the invention according to the seventh embodiment;

FIG. 18 of the drawing is an elevational view of safety syringe of the invention according to the eighth embodiment;

FIG. 19 of the drawing is an elevational view of safety syringe of the invention according to the ninth embodiment;

FIG. 20 of the drawing is an elevational view of safety syringe of the invention according to the tenth embodiment;

FIG. 21 of the drawing is a sectional view of safety syringe of the invention according to the eleventh embodiment;

FIG. 22 of the drawing is an elevational assembled view of safety syringe of the invention according to the twelfth embodiment;

FIG. 23 of the drawing is an elevational assembled view of safety syringe of the invention according to the thirteenth embodiment;

FIG. 24 of the drawing is an elevational assembled view of safety syringe of the invention according to the fourteenth embodiment;

FIG. 25 of the drawing is an elevational assembled view of safety syringe of the invention according to the fifteenth embodiment;

FIG. 26 of the drawing is an elevational assembled view of safety syringe of the invention according to the sixteenth embodiment;

FIG. 27 of the drawing is an elevational assembled view of safety syringe of the invention according to the seventeenth embodiment;

FIG. 28 of the drawing is an elevational assembled view of safety syringe of the invention according to the eighteenth embodiment;

FIG. 29 of the drawing is an elevational assembled view of safety syringe of the invention according to the nineteenth embodiment;

FIG. 30 of the drawing is an elevational assembled view of safety syringe of the invention according to the twentieth embodiment;

FIG. 31 of the drawing is an elevational assembled view of safety syringe of the invention according to the twenty-first embodiment;

FIG. 32 of the drawing is an elevational assembled view of safety syringe of the invention according to the twenty-second embodiment;

FIG. 33 of the drawing is an elevational assembled view of safety syringe of the invention according to the twenty-third embodiment;

FIG. 34 of the drawing is an elevational assembled view of safety syringe of the invention according to the twenty-fourth embodiment;

FIG. 35 of the drawing is an elevational assembled view of safety syringe of the invention according to the twenty-fifth embodiment;

FIG. 36 of the drawing is an elevational assembled view of safety syringe of the invention according to the twenty-sixth embodiment;

FIG. 37 of the drawing is an elevational assembled view of safety syringe of the invention according to the twenty-seventh embodiment;

FIG. 38 of the drawing is a sectional view of safety syringe of the invention according to the twenty-eighth embodiment;

FIG. 39 of the drawing is a sectional view of safety syringe of the invention according to the twenty-ninth embodiment;

FIG. 40 of the drawing is a sectional view of safety syringe of the invention according to the thirtieth embodiment;

FIG. 40A of the drawing is a sectional view of safety syringe of the invention according to the thirty first embodiment;

FIG. 41, FIG. 42, and FIG. 43 of the drawings are sectional views of safety syringe of the invention according to the thirtieth embodiment illustrating the sequence of operation of the syringe;

FIG. 44 of the drawing is a sectional view of safety syringe of the invention according to the thirty-third embodiment which illustrates the motion of the syringe;

FIG. 45 of the drawing is a sectional view of safety syringe of the invention according to the thirty-third embodiment;

FIG. 45A of the drawing is a sectional view of safety syringe of the invention according to the thirty-fourth embodiment;

FIG. 46 and FIG. 47 of the drawings are sectional views of safety syringe of the invention according to the thirty-third embodiment illustrating the sequence of operation of the syringe;

FIG. 48 of the drawing is a sectional view of safety syringe of the invention according to the thirty-fifth embodiment;

FIG. 49 of the drawing is a sectional view of safety syringe of the invention according to the thirty-sixth embodiment;

FIG. 50 of the drawing is a sectional view of safety syringe of the invention according to the thirty-seventh embodiment;

FIG. 51 of the drawing is a sectional view of safety syringe of the invention according to the thirty-eighth embodiment;

FIG. 52 of the drawing is a sectional view of safety syringe of the invention according to the thirty-ninth embodiment;

FIG. 53 of the drawing is a sectional view of safety syringe of the invention according to the fortieth embodiment;

FIG. 54 of the drawing is a sectional view of safety syringe of the invention according to the forty-first embodiment;

FIG. 55 of the drawing is a sectional view of safety syringe of the invention according to the forty-second embodiment;

FIG. 56 of the drawing is a sectional view of safety syringe of the invention according to the forty-third embodiment;

FIG. 57 and FIG. 58 of the drawings are sectional views of safety syringe of the invention according to the forty-third embodiment illustrating the sequence of operation of the syringe;

FIG. 59 of the drawing is a sectional view of safety syringe of the invention according to the forty-fourth embodiment;

FIG. 60 of the drawing is a sectional view of safety syringe of the invention according to the forty-fifth embodiment; and

FIG. 61 of the drawing is a sectional view of safety syringe of the invention according to the forty-sixth embodiment.

### DETAILED DESCRIPTION OF THE PREFERRED EMBODIMENT

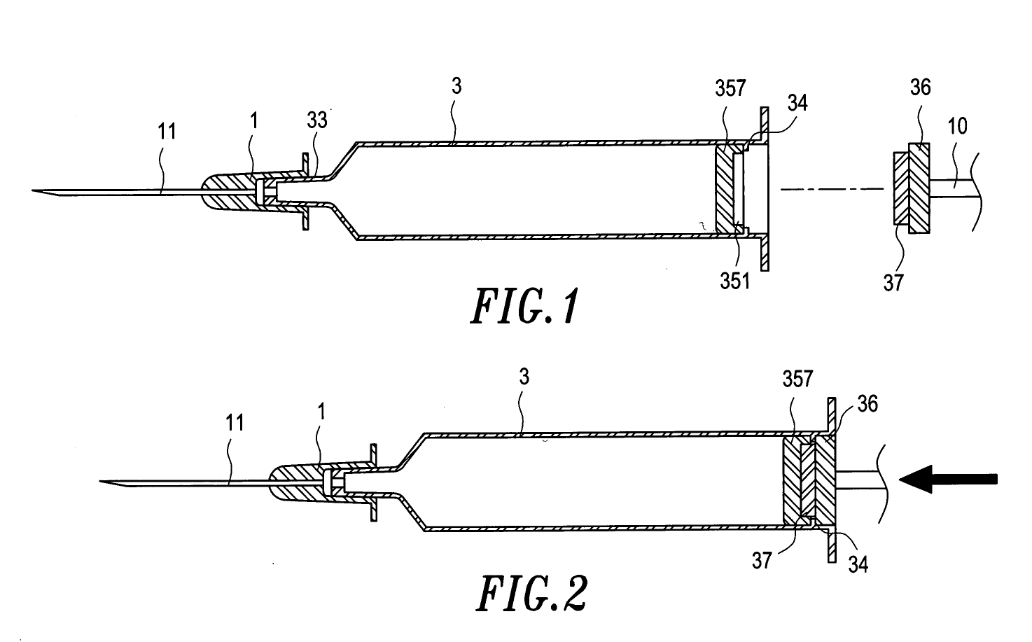

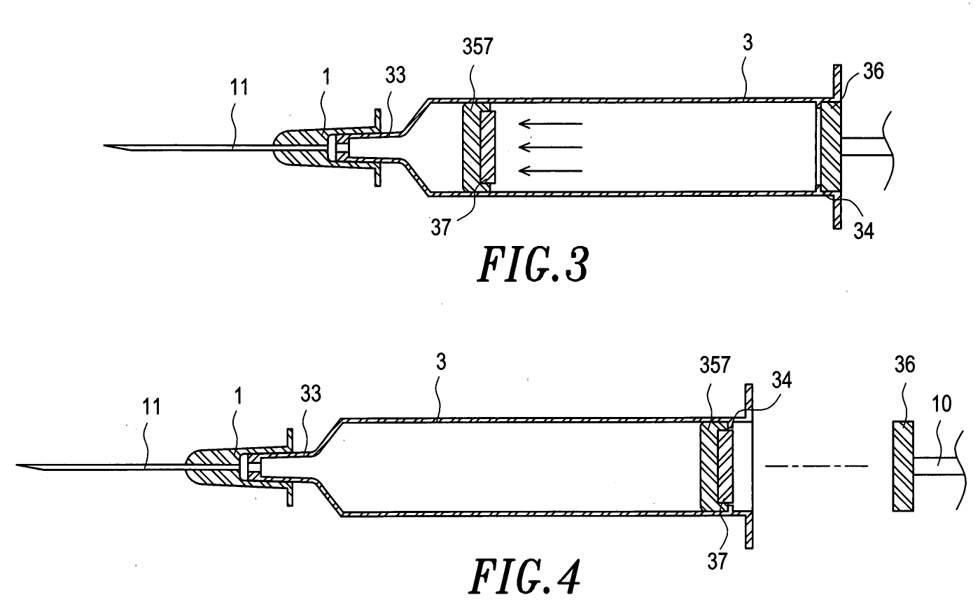

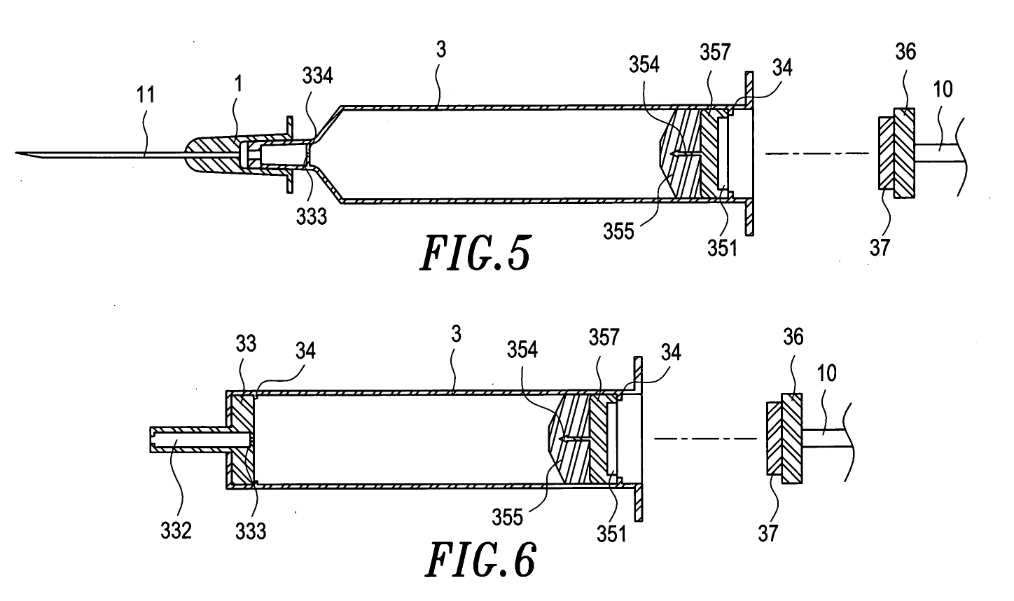

With reference to FIG. 1, FIG. 2 and FIG. 3, the present invention of safety syringe having:

a barrel 3, wherein a rubber stopper 357 with a shallow disk indention 351 at the rear end is inserted into the interior of the barrel 3, having a structure of annular check rib 34 formed in the rear portion of the inner surface of the hollow barrel 3, which restrains the rubber stopper 357 from leaving from the rear of the barrel 3, possessing a needle holder 33 engaging with a needle body 1 infused with the needle cannula 11; and

an actuator 10 comprising an electromagnet 36 and a magnet 37 opposite to one another with unlike poles attraction.

The method making the operable present invention of safety syringe includes the steps thereinafter. Firstly, inserting actuator 10 from the distal open end of the barrel 3, then, positioning the magnet 37 on the front surface of the actuator 10 that mates with the circular indention 351 on the rear surface of the rubber stopper 357, and finally switching the pole of electromagnet the same as the pole of the magnet 37 to induce the magnetic repellent force between the electromagnet 36 and the magnet 37 to compel the magnet 37 and the rubber stopper 357 forward by virtue of the law that opposite poles attract and like poles attract. After the rubber stopper 357 is compelled to the foremost end inside the barrel 3, the operator inserts the needle cannula 11 into patient's injection site. Thereafter, the pole of the electromagnet 36 is switched to be the unlike pole of the magnet 37, according to the foregoing law, the magnet 37 and the rubber stopper is attracted backward and the fluid of specimen (e.g., blood, dose, cell, human tissue, etc.,) is drawn through the cannula of the needle cannula 11 to the barrel 3. After the end of the injection or aspirating, the healthcare worker switches the pole of the electromagnet 36 and amplifies the induced magnetic force to attract the magnet 37 disengaging from the circular indention 351 of the rubber stopper 357 to the electromagnet 36 again. Henceforth, the actuator 10 can be removed from the open rear end of the barrel 3 now and the rubber stopper 357 stays there due to the restriction and locking structure of the annular check rib 34 formed at the rear end of inside surface of the barrel 3. As described above, the present invention of safety syringe utilizes the attraction and repellence mechanism between the electromagnet 36 and the magnet 37 of the actuator 10 to drive and pull the rubber stopper 357 reciprocally for injection or aspirating purpose, thereby this present invention of safety syringe cannot be used by the worker without the actuator 10 on hand. It is obvious that the foregoing mechanism reduce the likelihood of the second use of the used syringe and cross-infection. Additionally, as can be seen in FIG. 4, the magnet 37 is designed directly at the rear end of the rubber stopper 357 instead of attaching on the front surface of the actuator 10.

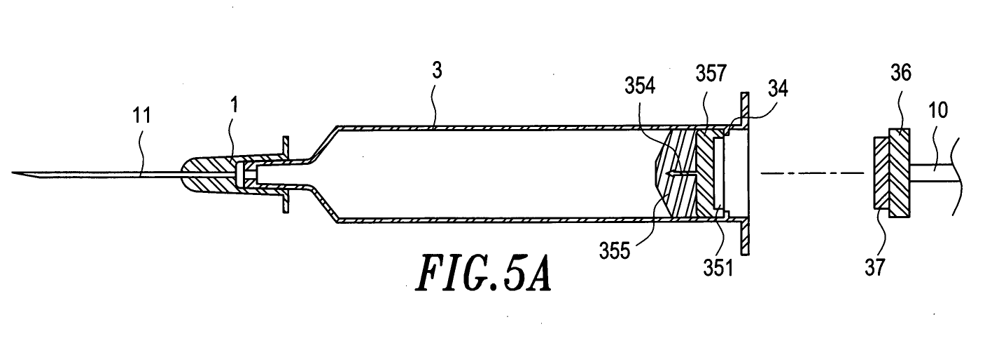

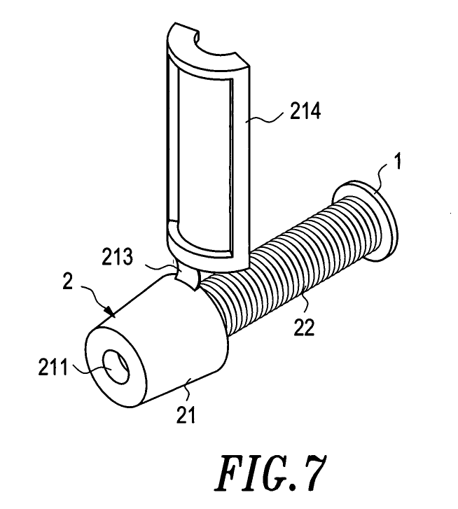

As can be illustrated in FIG. 5, the present invention of safety syringe has a circular check plate 334 with a through channel 333 in it. The spear-like barb 354 at the front end of the rubber stopper 357 is surrounded with the rubber wrap 355. After the end of injection or aspirating, the pole of the electromagnet 36 is switched in common with the magnet 37. Then, the increasing induced magnetic force of electromagnet drives the magnet 37 and the rubber stopper 357 to reach the foremost end of the barrel 3. In the mean time, the spear-like barb 354 pierces through the rubber wrap 355 and continuously squeeze over the through channel 333 and be lockingly engaged with the circular check plate 334. The objective of the structure of the spear-like barb 354 locking with the circular check plate 334 is to restrict the backward motion of the rubber stopper 357 so as to prevent the second use of said syringe. In addition, an alternative design is shown in FIG. 6 that circular check plate 334 and the through channel 333 are removed from the structure of the needle holder 33.

With reference to FIG. 6, the needle holder 33 is structured embedded in the barrel 3, the locking lumen 332 is formed inside the needle holder 33, the through channel 333 is used to communicate the locking lumen 332 with the interior of the barrel 3, and a annular check rib 34 is formed behind the needle holder 33 inside the barrel 3. Therefore, after the end of injecting and aspirating, the spear-like barb 354 is compelled to pierce through the rubber wrap 355 and continuously through the through channel 333 to the locking lumen 332 by resetting the pole of electromagnet 36 to be identical to that of magnet 37 and intensifying the magnetic field of the electromagnet 36. At this moment in time, the needle holder 33 retracts into the barrel 3 and breaks through the annular check rib 34 by means of switching the pole of the electromagnet 36 opposite to magnet 37 and intensifying the magnetic field of electromagnet to increase the magnetic attraction force of the electromagnet 36 and the magnet 37.

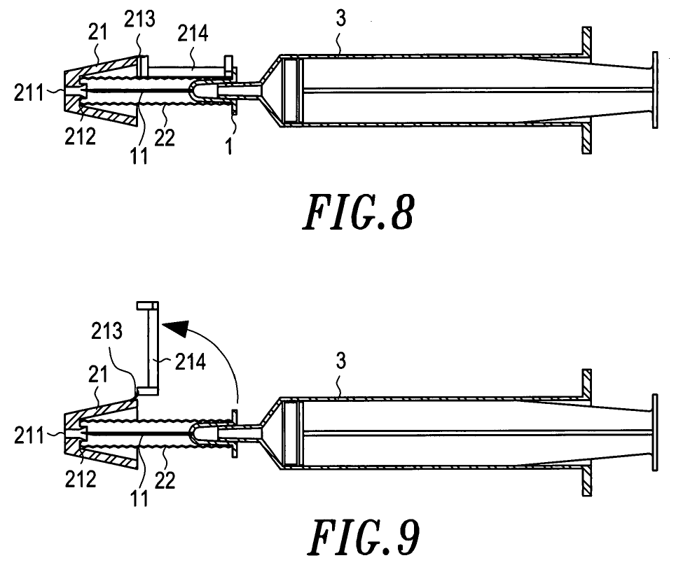

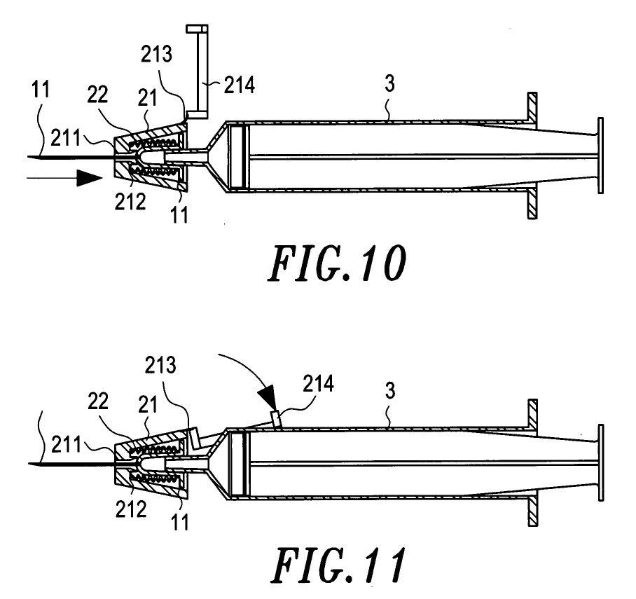

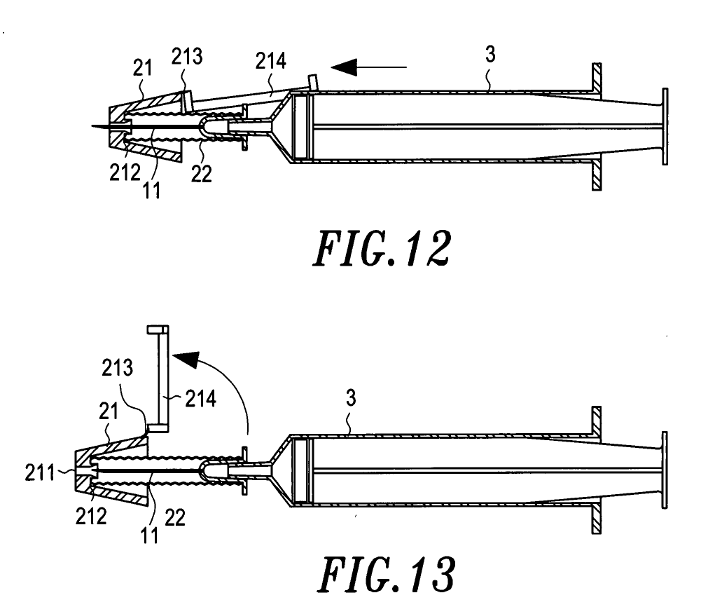

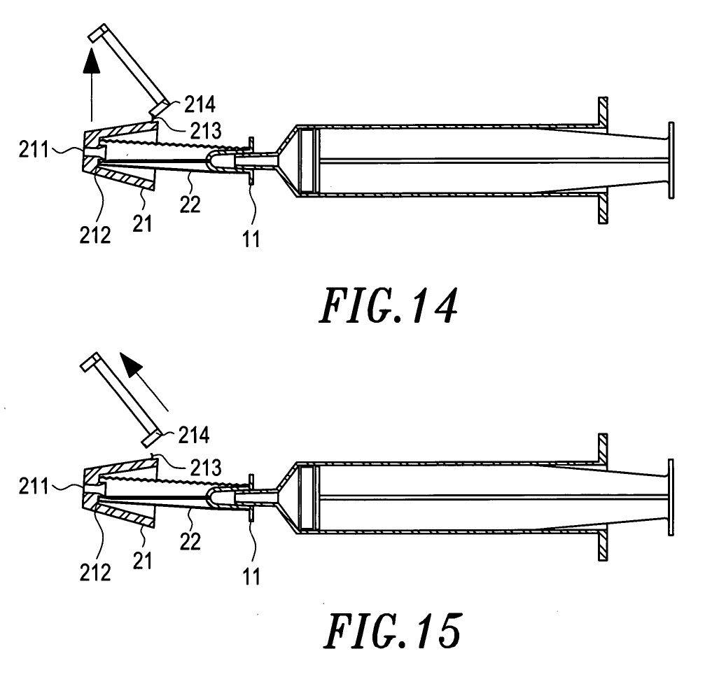

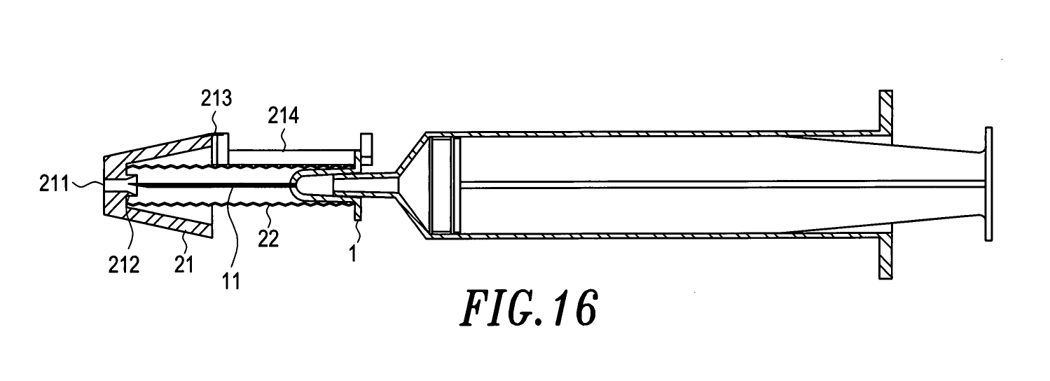

As can be shown from FIG. 7 to FIG. 16, the present invention of safety syringe, more particularly, refers to a device of safety mechanism mainly including:

a needle body 1, the needle cannula 11 is fused at the front end of the needle body; and

a needle shield 2, which is comprised of a shield base 21 and a retractable elastomeric tube 22, wherein a needle passageway 211 is constructed in the front end of the shield base 21 and a nozzle-like circumferential check plate 212 is formed adjacent to the needle passageway 211 of the shield base 21, which has a connection tab 213 in linkage to a buttoning check plate 214 and the retractable elastomeric tube 22 to connect the shield base 21 with the needle body 1.

The process for making the present operable invention of safety syringe comprises the steps thereinafter. First, healthcare professionals mate the needle body 1 with the barrel 3, secondly, detach the buttoning check plate 214 from the needle body 1 and pivot about the connection tab 213, thirdly, pull both of the buttoning check plate 214 and the shield base 21 backward, wherein the retractable elastomeric tube 22 is contracted in the containing space of the shield base 21, and fourthly uncover the tip portion of the needle cannula 11 fused in the needle body 1 through the needle passageway 211 of the shield base 21. Henceforth, healthcare workers administer injection or aspirating. After the end of the injection or aspirating, the operator only needs to push the buttoning check plate 214 with the shield base 21 forward to cover the needle cannula 11, pulls the buttoning check plate 214 in the direction perpendicular to the needle cannula 11 to shift the needle tip of the needle cannula 11 over the nozzle-like circumferential check plate 212 and trap the needle cannula 11 in the space formed of the inner face of shield base 21 and the nozzle-like circumferential check plate 214. This trapping mechanism inhibits the needle cannula 11 from being uncovered through the needle passageway 211 once again. Moreover, while the healthcare worker is pulling the buttoning check plate 214 in the direction perpendicular to the needle cannula 11 to trap the needle tip, they can pull heavily to break through the connection tab 213 and detach the buttoning check plate 214 from the shield base 21. The present invention provides a safety syringe in injection or aspirating operation hereinabove, the healthcare professional's hand can manipulate behind the tip of the needle cannula 11 throughout the process to avoid the risk of needlestick injuries. Furthermore, after the end of injection or aspirating, the present invention of safety syringe is incapable of being reused because the trapping mechanism formed of the shield base 21 and the nozzle-like circumferential check plate 212 inhibit the needle cannula 11 from uncovering through the needle passageway 211 again.

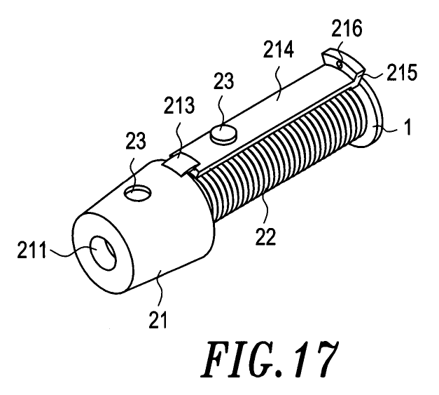

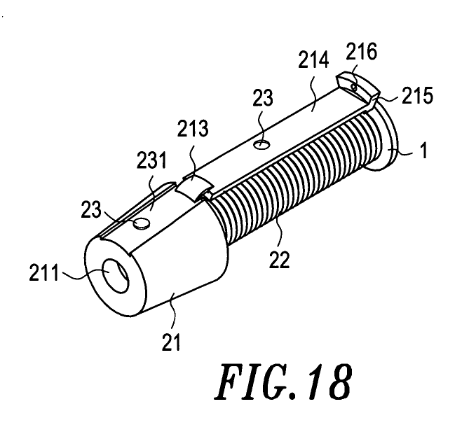

With reference to FIG. 17 and FIG. 18, in the present invention of safety syringe, a button structure 23 formed as a part on the outer surface of the shield base 21 and the outer side of the buttoning check plate 214 respectively is designed in lieu of the nozzle-like circumferential check plate 212 adjacent to the shield base 21 and the needle passageway 211. An upstanding seat 215 with a needle tip trapping pit 216 at its front end rim surface is formed. After the end of injection and aspirating, the healthcare workers need not to push both of the buttoning check plate 214 and the shield base 21 ahead to cover the needle cannula 11, but to pivot the buttoning check plate 214 about the connection tab 213 counterclockwise and lock the button structure 23, which is located on the outer side of the shield base 21 and the outer surface of the buttoning check plate 214. At this moment in time, the needle tip of the needle cannula 11 is trapped and engaged in the needle tip trapping pit 216 positioned in the upstanding seat at the rear end of the buttoning check plate 214, because the button structure 23 positioned on the outer side of the shield base 21 and the corresponding edge of the buttoning check plate 214 is in the dead lock condition. Consequently, it is obvious that the buttoning check plate 214 firmly engages with the shield base 21. That prevents the present invention of safety syringe from being reused. Furthermore, an alternative design of a buttoning rectangular space 231 of shield base 21, where the buttoning check plate 214 is mating with, is formed in lieu of the button structure 23.

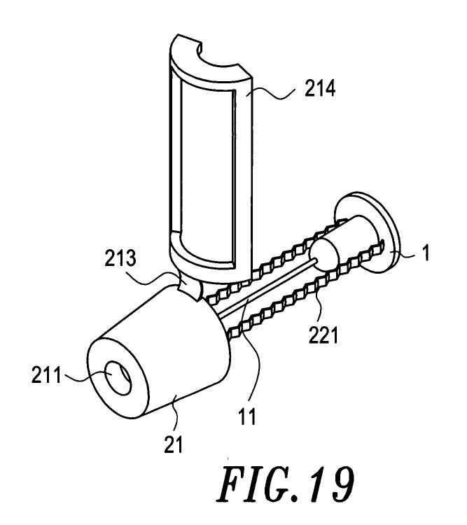

With reference to FIG. 19 and FIG. 20, in the present invention of safety syringe, two lamented leaf springs 221, 221 positioned in the outer side of needle cannula 11 diametrically opposite to each other is designed instead of the retractable elastomeric tube 22. Furthermore, an alternative design similar to the foregoing approach is made of mounting and penetrating the needle cannula 11 within a sequence of guided holes 222 on a specifically designed lamented leaf spring 221.

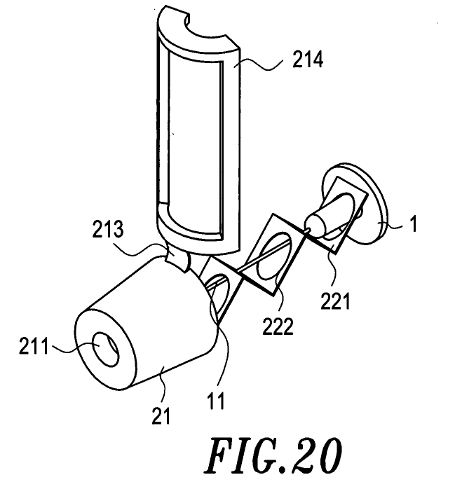

Referring to FIG. 21, the shield base 21 is miniaturized to be positioned appositely in the outer side of the needle body 1, the specifically designed lamented leaf spring 221 is compressed inside the space between the shield base 21 and the needle holder, and the outer rim of the needle cannula 11 is still covered within a needle cap 2. While the present invention of safety syringe is used, the needle cap 2 is removed from said syringe to administer the injection or aspirate. After the end of injection and aspirating, the shield base 21 is pushed forward and the specifically designed lamented leaf spring 221 shields the needle cannula 11. The buttoning check plate 214 is pulled in the direction vertical to the needle cannula 11 thereupon and forced the needle tip of the needle cannula 11 to transverse over the nozzle-like circumferential check plate 212 into; the locking space positioned between the interior of shield base 21 and the nozzle-like circumferential check plate 212. Finally, the needle cannula 11 is restrained from the foregoing locking structure to avoid another penetration through the needle passageway 211, and in the mean time, the shield base 21 is incapable of retraction due to the needle cannula 11.

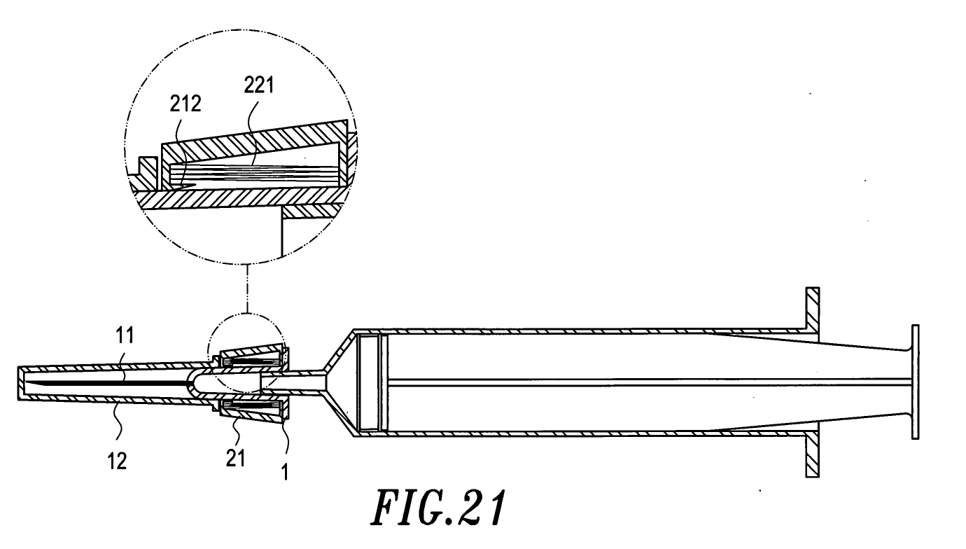

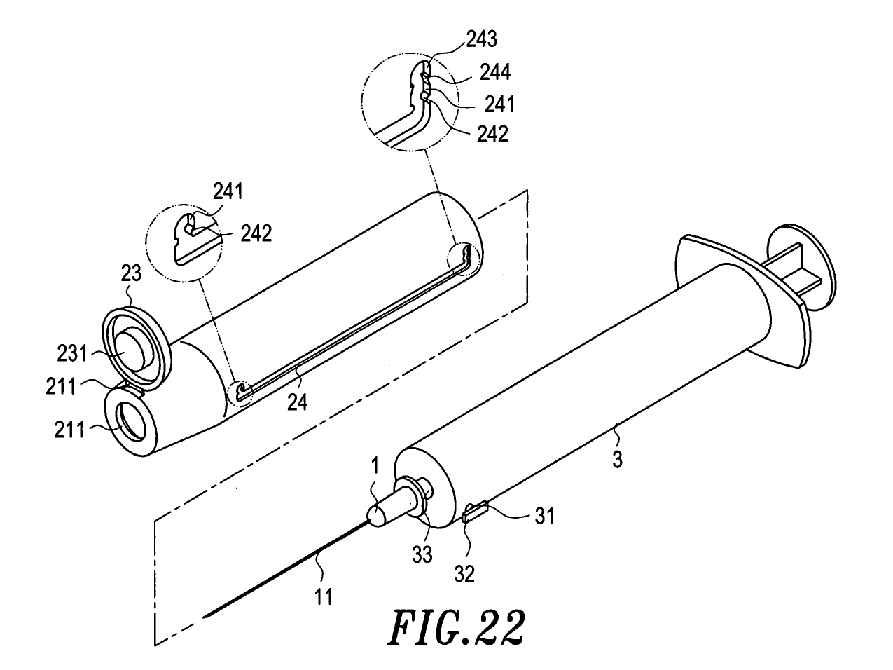

With reference to FIG. 22, the present invention of safety syringe, more particularly, proposes a safety structure of the barrel 3 having:

a needle body 1 wherein the needle cannula 11 is fused;

A cap 2 having a needle passageway 211 at the front end, a connection tab 32 connecting with the cap 23 and the cap 2, a inner projection bung 231 inside the inner face of the front cap 23, two guided slots 24, 24 positioned diametrically in opposite side of the exterior of the cap 2, a front and a rear end containing cut 241 and the anchor cut 243 of the guided slot 24, and a plurality of V-shape check ledges 244 between the anchor cut 243 and the rear end containing cut 241; and

a barrel 3 having two sliding rods 31, 31 with ear tabs 32, 32 on opposite side of the barrel 3.

The method of making the present operable invention of safety syringe includes the steps thereinafter. In the beginning, the needle body 1 is engaged with the front end of the needle holder 33, and the two guided sliding rods 31, 31 on the opposite side of the barrel 3 are positioned at the rear end containing cut 241 of the guided slot 24. Secondly, healthcare workers disclose the front cap 23, twist to loose of the anchor cut 243 of the cap 2, and lead the guided sliding rods 31 to depart from the anchor cut 243 across the V-shape check ledge 244 to the rear end containing cut 241 and finally go through the ledge 242 to the guided slot 24. The operator holds the ear tab 32, and thereupon pulls the cap 2 rearwards to uncover the needle cannula 11 through the needle passageway 211. Henceforth, the operator twists the cap 2 and mates the guided sliding rod 31 with the front side containing cut 241 crossing over the ledge 242. At this moment in time, the healthcare professionals can administer the injection or aspirate. After using this syringe for its intended procedure, the operator twists and looses the cap 2 to position the guided sliding rod 31 to the guided slot 24, and pushes the cap 2 forward. From that time on, the guided sliding rod 31 is mated with the rear side containing cut 241 crossing over the ledge 242, and the cap 2 is twisted to adapt the guided rod 31 to cross over the V-shape check ledge 244 and engage into the anchor cut 243 connected to the rear end of the guided slot 24. At last, the healthcare professional completes the operation to recap the front cap 23 into the front end of the cap 2. As a result, the present invention of safety syringe is incapable of being reused by virtue of the mechanism of the V-shape check ledge 244 to restrain the guided sliding rod 31 from dislodging the anchor cut 243. Additionally, an alternative approach, as can be seen in FIG. 23, is proposed to position two guided sliding rods 31, 31 and two ear tabs 32, 32 diametrically in the opposite side of the interior of the cap 2. Two opposite guided slots 24, 24 are formed in the outer surface of the barrel 3 of the syringe. Two containing cuts 241, 241 are positioned at both of the ends of the guided slot 24, a plurality of ledges 242 are made between the containing cut 241 and guided slot 24, and a multiple of V-shape check ledges 244 are formed between the containing cut 241 and the anchor cut 243 at the front end of the guided slot 24.

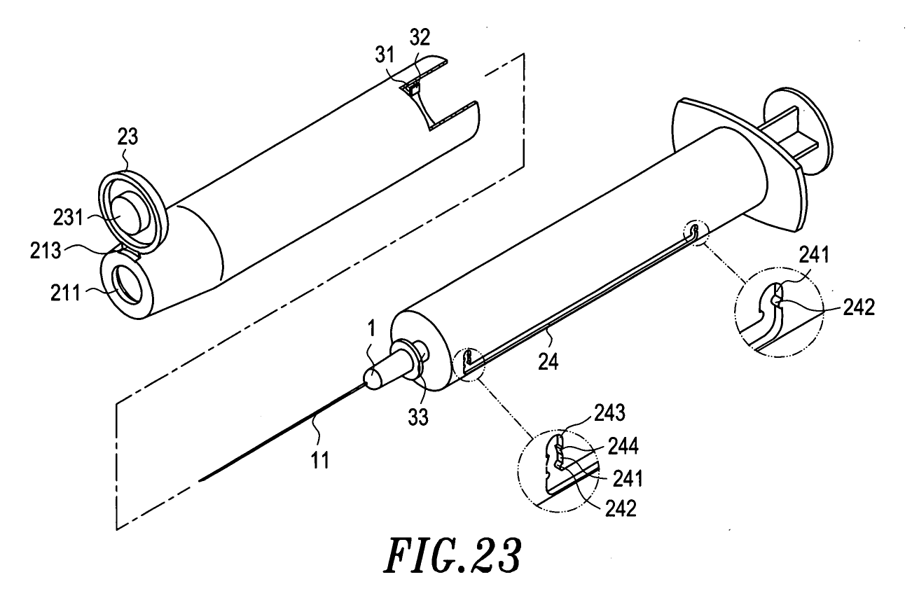

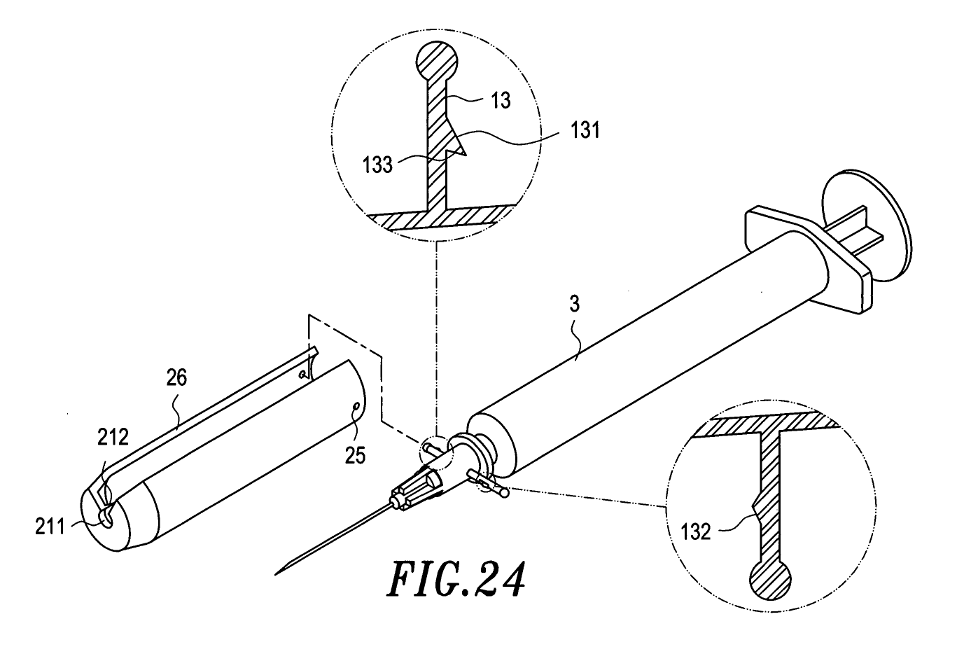

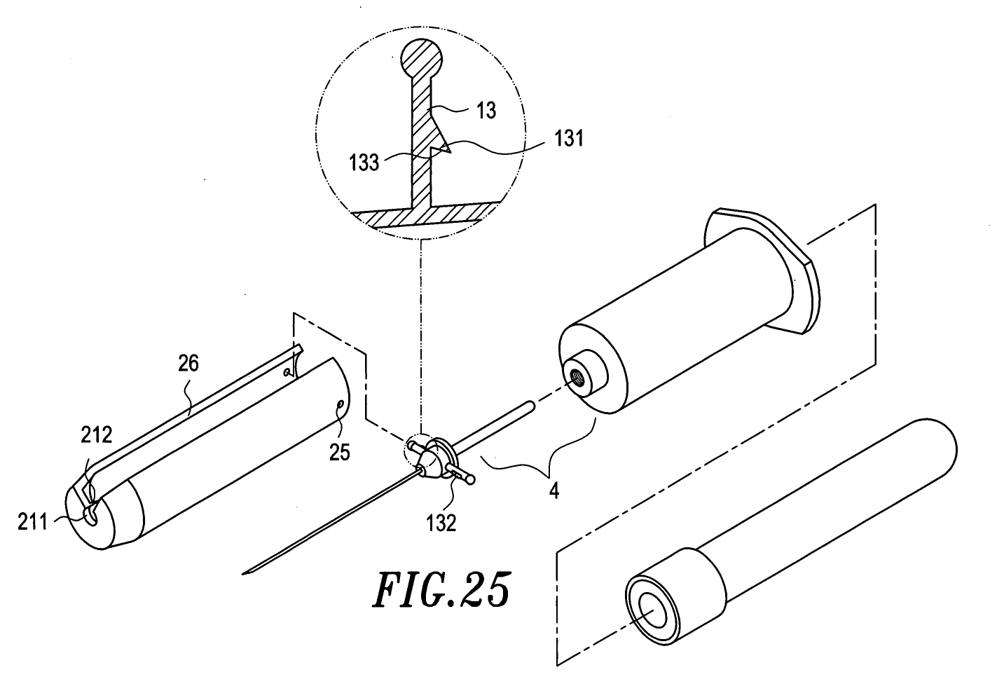

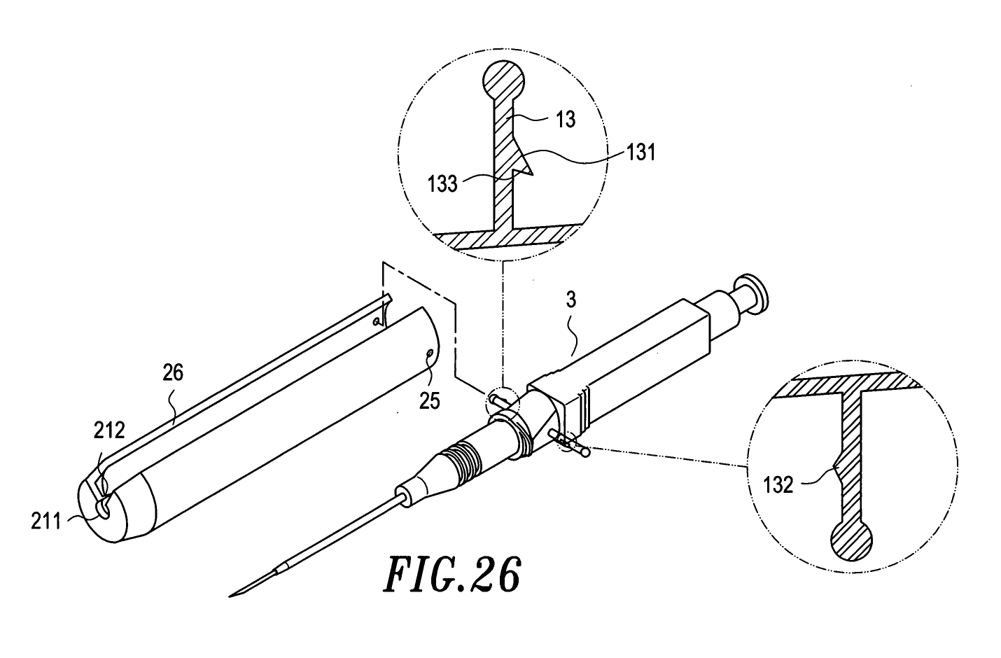

With reference to FIG. 24, for another alternative design in the present invention of safety syringe, two spindles 13, 13, two mounting holes 25, 25, an opening cutout 26 and a nozzle-like circumferential check plate 212 are designed instead of the guided slot 24 of the cap 2, the front cap 23, and the ear tab 23 in the outer side of the barrel 3. These two spindles 13, 13 are positioned diametrically in opposite side on the circumference of the needle holder, where one spindle 13 is structured with a positioning bump 131 in shape of a barbed acute angle heading in the direction towards the axis and a bias angle 133 between the bump side and spindle side near the syringe, and the other spindle 13 is structured with an adjusting bump 132 in the shape of obtuse angle head. Those two mounting holes 25, 25 positioned diametrically opposite to one another in the rear portion of the cap 2 are corresponding to these two spindles 13, 13. The opening cutout 26 is made between two mounting holes 25, 25. The nozzle-like circumferential check plate 212 is made behind the needle passageway 211. When the present invention of safety syringe is used, firstly, the cap 2 is pivoted about the spindle 13 to uncover the needle cannula 11 through the opening cutout 26 for the injection or aspirating. After the end of injection and aspirating, the cap 2 is pivoted back to the original covering position, and then the operator pushes the front portion of the cap 2 towards the positioning bump 131 of the spindle 13 to squeeze the positioning bump 131 through the mounting hole 25. It is obvious that the outer wall of cap 2 engaging with the side of bias angle 133 leads the cap 2 and the needle body 1 to be eccentric and results in the tip of the needle cannula 11 crossing over the nozzle-like circumferential check plate 212 and being trapped. In addition, the foregoing mechanism can be applied in the blood collection system 4, the infusion set needle 5, the arterial venous fistula needle 6 and the butterfly injection needle 7, as can be referred to FIG. 25, FIG. 26, FIG. 27 and

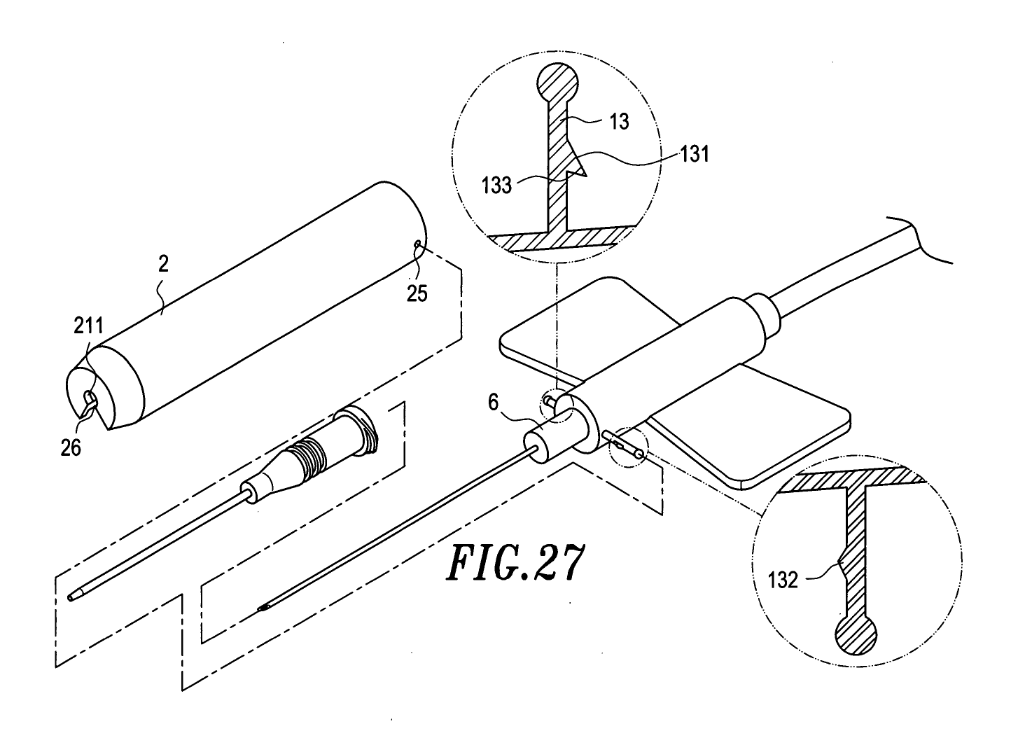

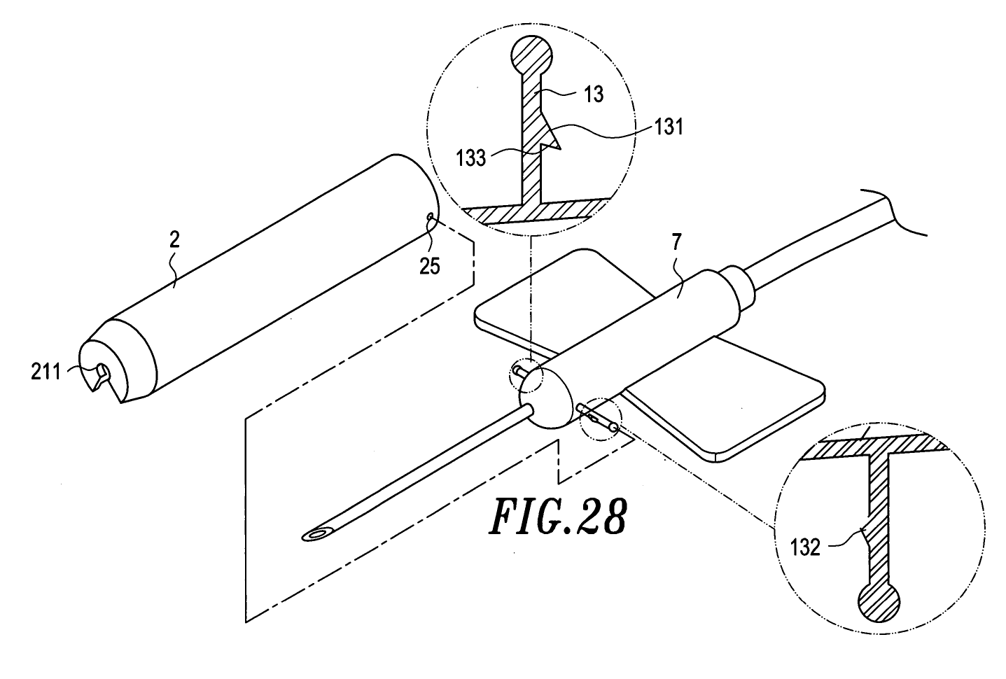

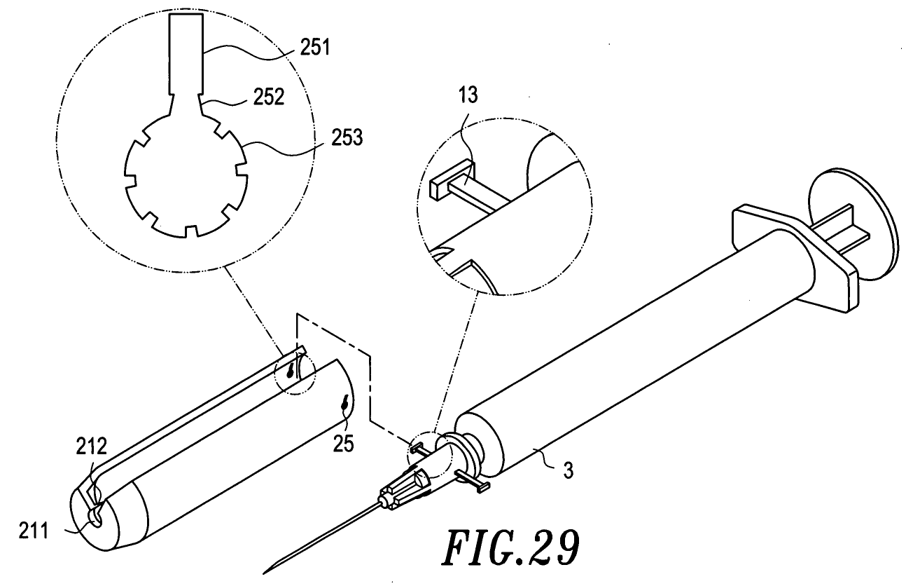

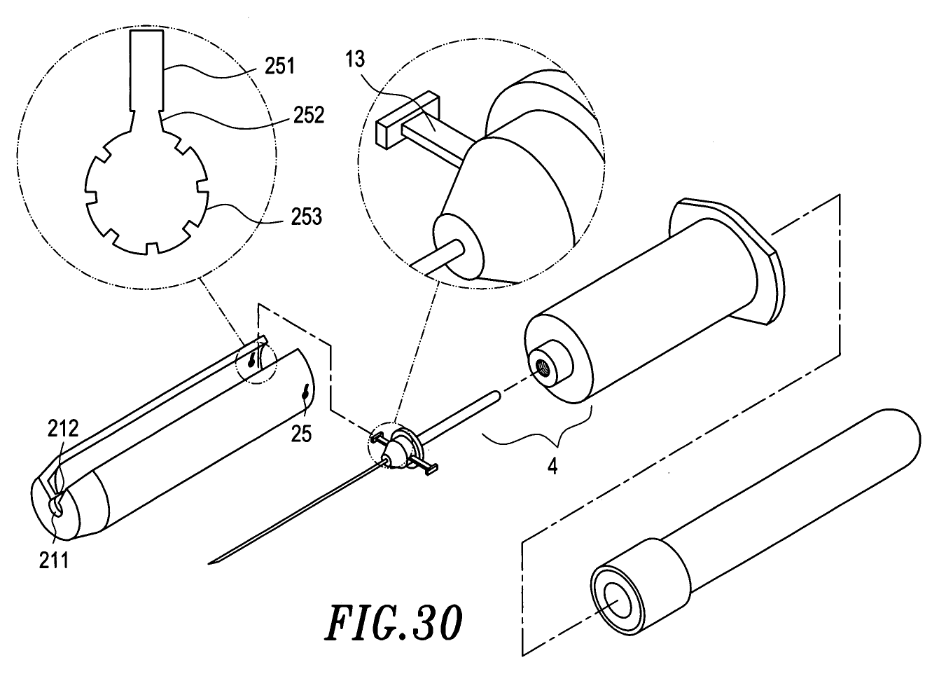

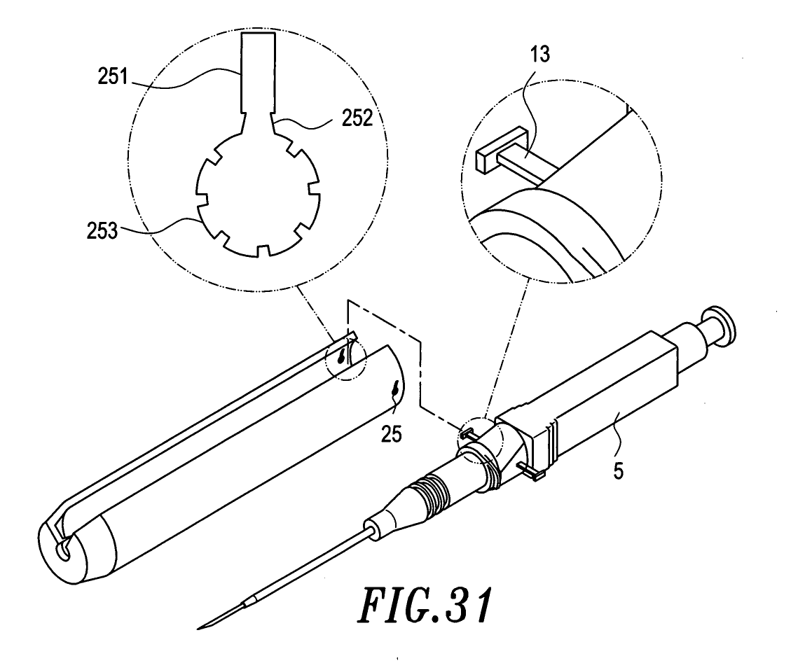

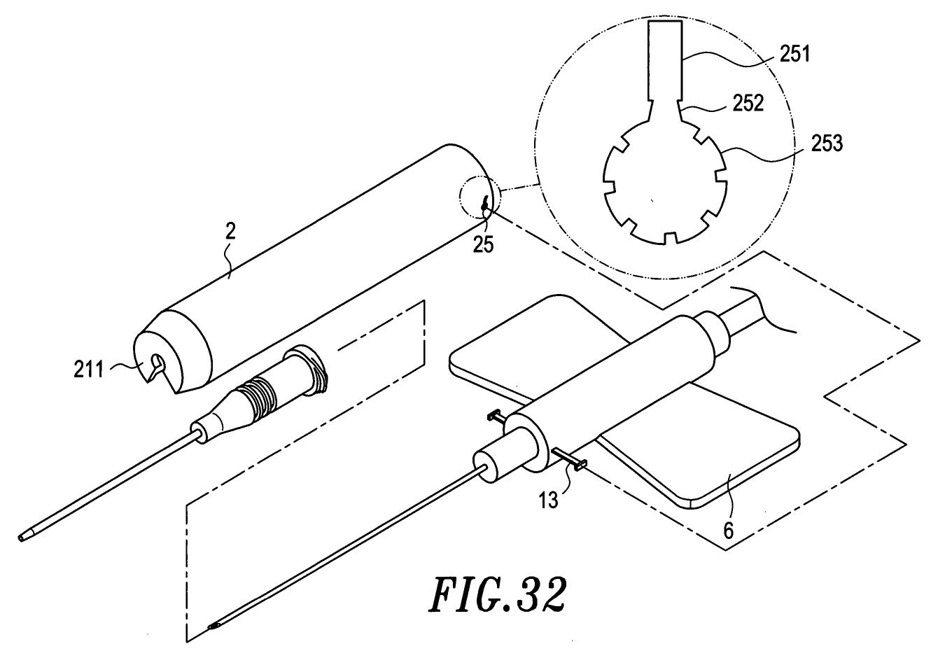

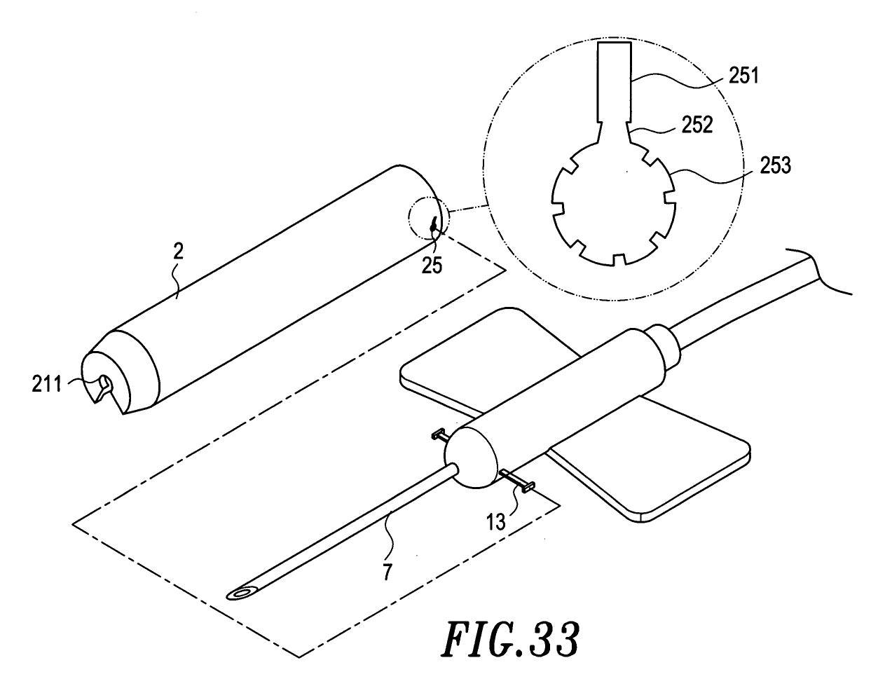

FIG. 28. Otherwise, an alternative approach instead of the positioning bump 131 and the adjusting bump 132 is proposed hereinafter. The mounting hole 25 is connected with a rectangular anchor cut 251 and the portion between the mounting hole 25 and the rectangular anchor cut 251 is structured with a multiple of check parts 252. Inside the mounting hole 25, there are a plurality of angle positioning broached contours 253 designed to position the required angle while the cap 2 is pivoted about the spindle 13, as can be seen in FIG. 29. After the end of injection or aspirating, the spindle 13 of the cap 2 is driven to position in the rectangular fixing cut 251. To avoid the risk of reuse, the spindle is designed to be incapable of escaping from the rectangular fixing cut 251 by means of the restriction of the check parts 252. Additionally, the foregoing mechanism can be applied in the blood collection system 4, the infusion set needle 5, the arterial venous fistula needle 6 and the butterfly injection needle 7, as can be referred to FIG. 30, FIG. 31, FIG. 32 and FIG. 33.

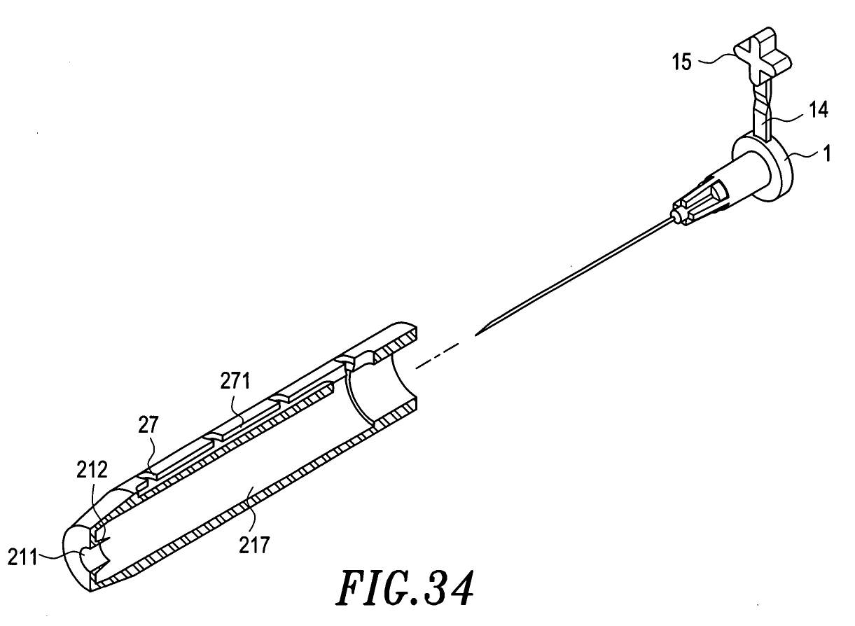

With reference to FIG. 34, in the present invention of safety syringe, the upstanding notch connector 14 with the cross sliding adjustor 15 in the radially outer side and the needle body 1 in the radially inner side are used instead of the front cap 23, the guided slot 24, and the guided sliding rod 31. A plurality of positioning slots 27 are arranged longitudinally in line on the circumferential surface of the cap 2. These positioning slots 27 are connected with a connection groove 271, except the first positioning slot 27 from the rear end of the cap 2 is directly connected to the needle containing cavity/housing 217 inside the cap 2. Furthermore, a nozzle-like circumferential check plate 212 is structured behind the needle passageway 211 in the cap 2. And the profile of the exterior of the cap 2 and the needle containing cavity/housing 217 are eccentric. Prior to use the safety syringe, the cross sliding adjustor 15 is initially positioned in the second positioning slot 27 from the rear end of the cap 2 and the needle cannula 11 is shielded in the cap 2. Then, the healthcare professionals dislodges the cross sliding adjustor 15 from the positioning slot 27, pull the cap 2 rearwards, move the upstanding notch connector 14 towards the front end of the connection groove 271 to protrude the needle cannula 11 through the needle passageway 211 for a pertinent operating length, and reset the cross sliding adjustor 15 td the nearest apposite position slot 27 before the injection and aspirating. After the end of the injection and aspirating, the healthcare workers push the cap 2 ahead and reset the cross sliding adjustor 15 to the first positioning slot 27 from the proximal end of the cap 2. At the moment of this time, the cap 2 is driven foremost. By means of the eccentric of exterior of the cap 2 and the needle containing cavity/housing 217 as well as the position of the cap 2, the tip of needle cannula 11 is positioned over the nozzle-like circumferential check plate 212 and being trapped. Penetration through the needle passageway 211 is unlikely. Furthermore, an alternative design is proposed that the upstanding notch connector 14 is not positioned on the needle body 1, but on the exterior of a washer set 8. The washer set 8 is positioned and mated between the needle body 1 and the front end of barrel 3, as can be referred to FIG. 35. In addition, the foregoing cross sliding adjustor 15 can be in any shape with respect to the accommodation shape of the corresponding positioning slot 27 and the connection groove 271, as can be referred to FIG. 34 and FIG. 36.

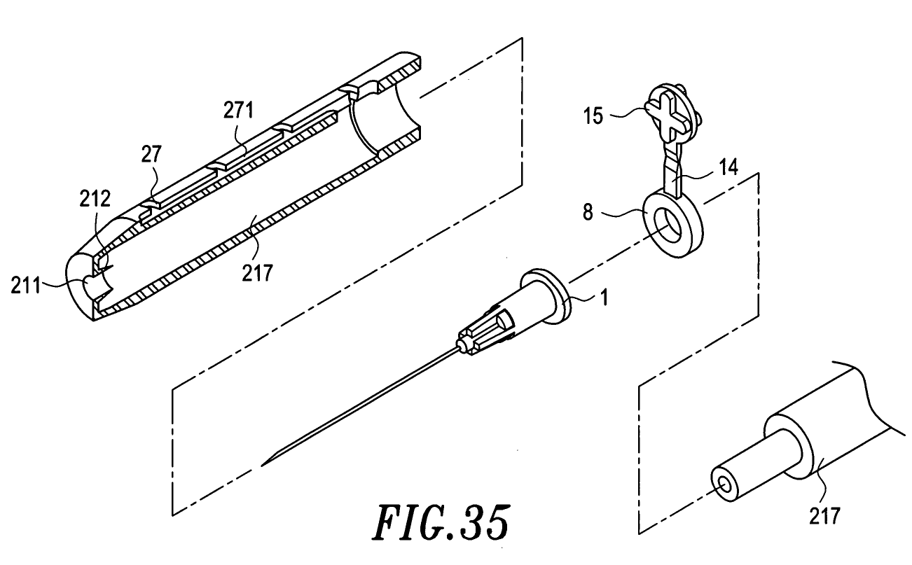

With reference to FIG. 37, in the present invention of safety syringe, an alternative design is proposed to use a positioning hole 16 in the side surface of the needle body 1 and a positioning adjustor 15 to replace the upstanding notch connector 14. The positioning hole 16 is formed of an anchor recess 161 at the radially inner end, a positioning cavity 163 in the middle, a transition cavity 164 at the radially outer end, and a plurality of annual check plates 162 around the passing opening on the top of the anchor recess 161. The positioning adjustor 15 has a positioning rod 151, a positioning ball 152 corresponding to those recess and cavities at the distal end therein and a check block 153 in the one or both sides of the positioning adjustor 15. Initially, the positioning adjustor 15 is positioned in the first positioning slot 27 from the rear end of the cap 2 and the positioning ball 153 at the distal end of the positioning rod 151 is positioned within the positioning recess 163. The healthcare worker only needs to vertically pull the positioning ball 153 with the positioning rod 151 to the transition cavity 164 to free the positioning adjustor 15 from the positioning slot 27, and after that push the cap 2 rearward to uncover the needle cannula 11 through the needle passageway 211 with an appropriate length, and reset the positioning adjustor 27 to the nearest available positioning slot 27. After the injection or aspirating, the healthcare professionals pull vertically the positioning ball 153 with the positioning rod 151 to the transition cavity to free the positioning adjustor 15 from the positioning slot 27, push the cap 2 ahead, reset the positioning adjustor 15 to the first positioning slot 27 from the rear end of the cap 2, and press the positioning ball 153 with the positioning rod 151 into the anchor recess 161. At the moment of this time, the positioning ball 153 is restrained from the annual check plate 162 between the anchor recess 161 and positioning cavity 163 as well as the check blocks 153 on one or both sides of the positioning adjustor 15 are engaged against the inner wall of the cap 2. That restricts the cap to move backward and provides the double-proof to avoid the second use of syringe.

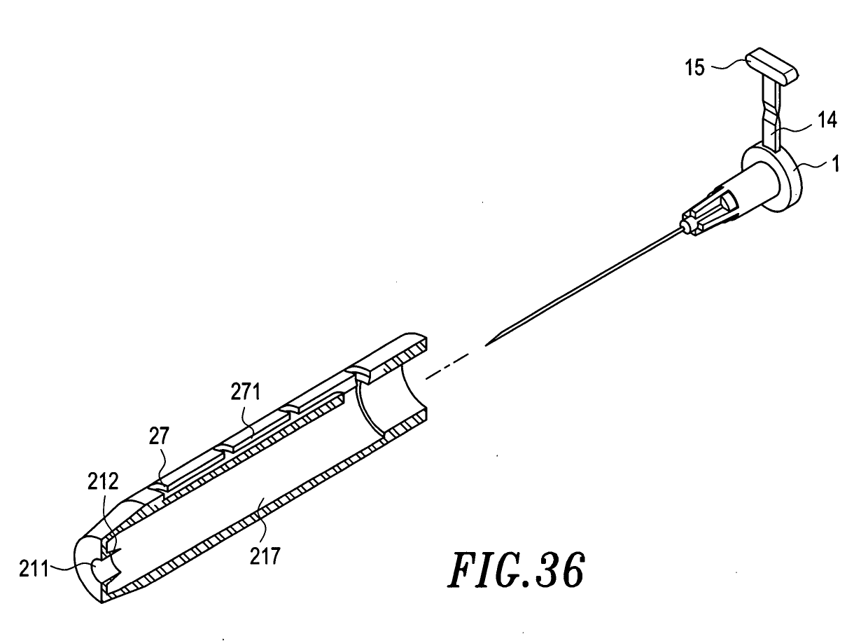

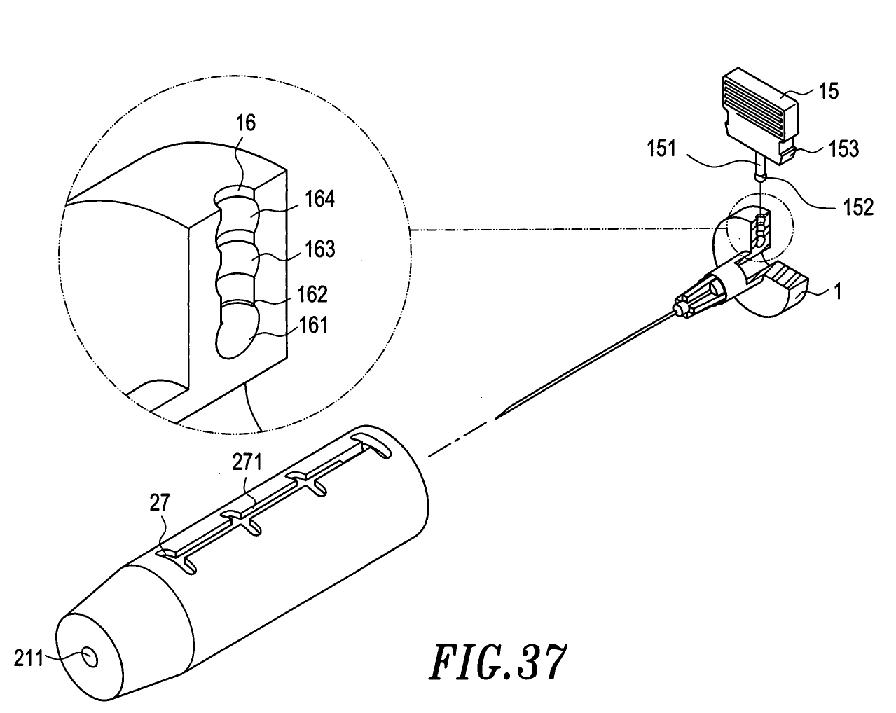

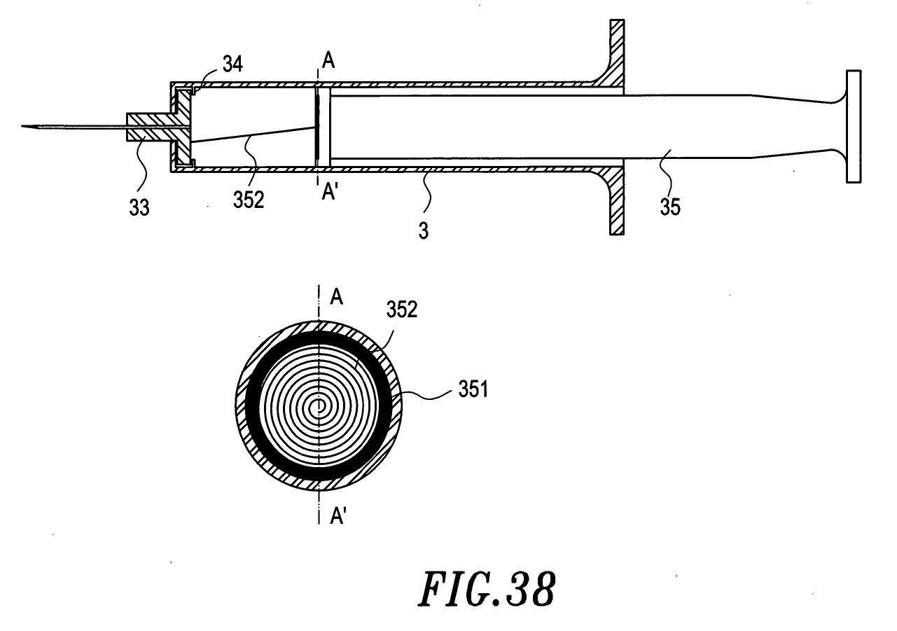

With reference to FIG. 38, the present invention of safety syringe having:

a barrel 3 having a needle seat 31 embedded in the front portion and an annual check rib 34 in the abutment to the needle seat 31;

a piston rod 35 with a linear elastomer groove 351 at the front end; and

a linear elastomer 352 which is connected around the linear elastomer groove 351 at one end and connected to the rear surface of the needle holder 33 at the other end.

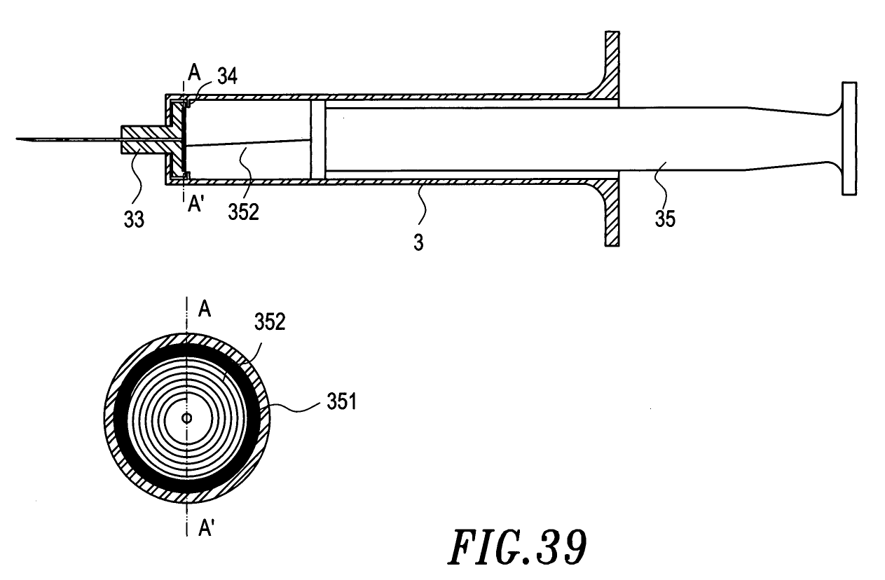

After the end of injection or aspirating, the healthcare professionals only need to pull the piston rod 35 backwards. The linear elastomer 352 connected around the linear elastomer groove 351 of the piston rod 35 is extended rearward with the piston rod 35 to its maximum limit. After that, the continuously pulling force coupling with the elastic force from the linear elastomer 352 brings the needle holder 33 backwards to break through the annual check rib 34 and enter inside the cavity of the barrel 3 to avoid the syringe for reuse. Referring to FIG. 39, an alternative design is to rearrange the linear elastomer groove 351 to the rear face of the needle seat and the linear elastomer 352 directly connected to the front surface of the piston rod 35.

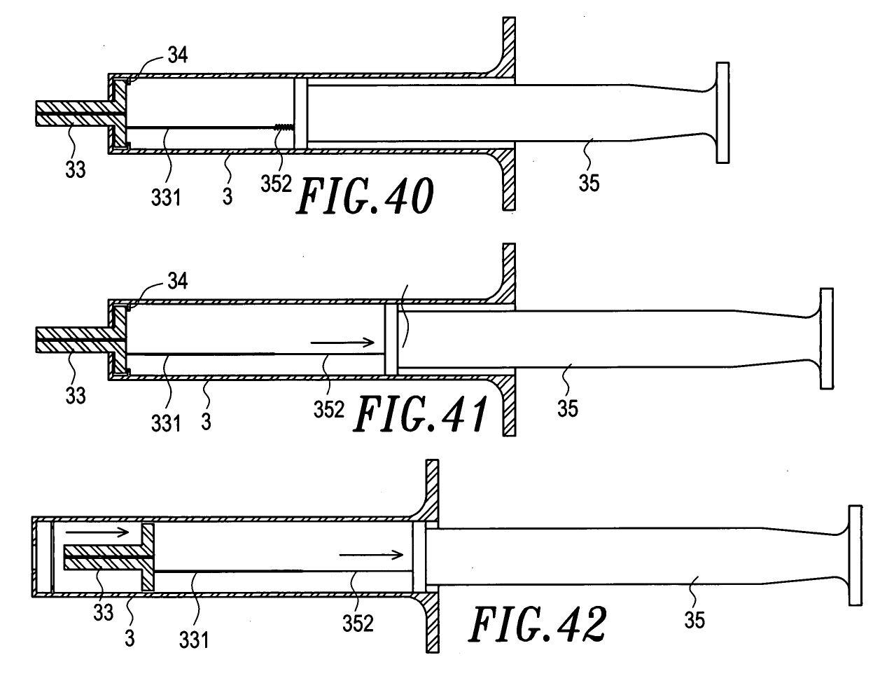

With reference to FIG. 40, FIG. 41, and FIG. 42, in the present invention of safety syringe, the alternative design is to replace the linear elastomer groove 351 with a linkage rod 331 directly formed at the rear end of the needle holder 33 which is connected to a linear elastomer 352 affixed to the front portion of the piston rod 35. The linear elastomer 352 connected to the linkage rod 331 can be extended rearwards with the piston rod 35 to its maximum limit. After that, the continuously pulling force coupling with the elastic force of the linear elastomer 352 brings the needle holder 33 backwards to break through the annual check rib 34 and enter inside the cavity of the barrel 3 to avoid this syringe for reuse. Moreover, as can be shown in FIG. 40A, the linkage rod 331 can be elongated through the piston rod 35 to be connected with a linear elastomer 352 linking to the front surface of a block 353 at another end. After injection and aspirating, by means of pushing the piston rod 35 backward to compress the linear elastomer 352 to force the block 353 springing out. Henceforth, the outward force coming from the block 353 brings the needle holder 33 into the interior of the barrel 3. Moreover, an alternative design is to use the linkage rod 331 in connection with the block 353 directly.

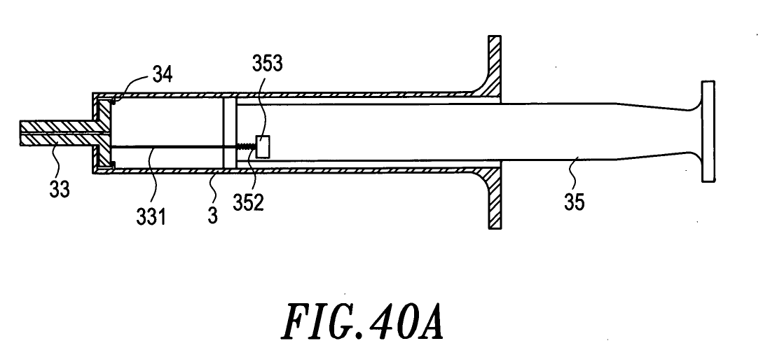

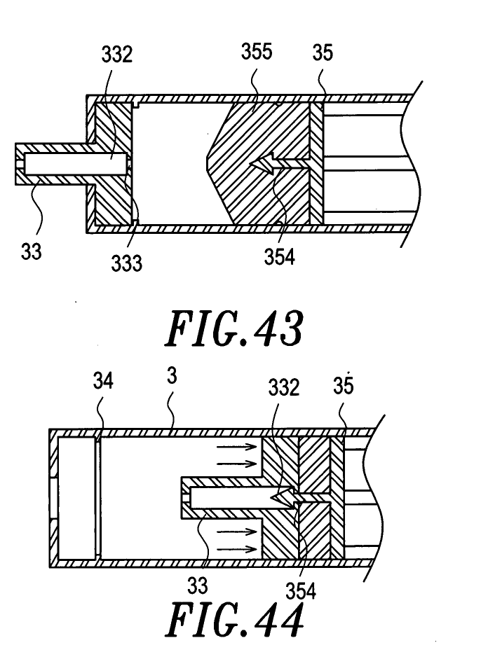

With reference to FIG. 43 and FIG. 44, in the present invention of safety syringe, an alternative design is proposed to replace the linear elastomer groove 351 with a spear-like barb 354 surrounded by a rubber wrap 355. A locking lumen 332 is formed inside the needle holder 33, which communicates the interior cavity with the through channel 333 in the rear portion of the needle holder 33. After the completion of the injection or aspirating, healthcare workers only need to push the piston rod 35 ahead to closely match the rubber wrap 355 with the profile of the rear wall of the locking lumen 332 and continuously push the piston rod 35 to coerce the spear-like barb 354 to pierce through the rubber wrap 355 and the through channel 333 to lock in the locking lumen 332. After that, the operators pull the piston rod 35 backward with the needle holder 33 and retreat the needle cannula 11 into the interior of the barrel 3.

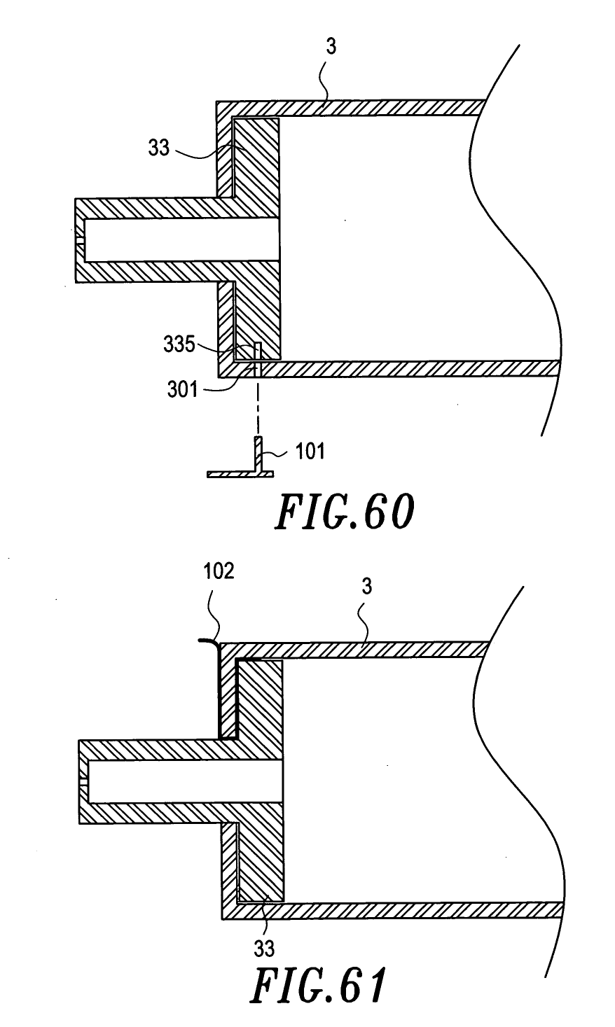

With reference to FIG. 60, in the present invention of safety syringe, a keyway through hole 301 formed in the front portion of the side wall of the barrel 3, a keyway hole 335 formed in the position of the connection mount of the needle holder 33 corresponding to the keyway through hole 301, and a pin key 100 inserted through the keyway through hole 301 and keyway hole 335 to engage the connection mount of the needle holder 33 with the front portion of the barrel 3 are proposed in lieu of the annular check plate 34 formed as a part of inner surface of the barrel 3 adjacent to the connection mount of the needle holder 33. After using the syringe for its intended procedure, the healthcare professional only needs to pull out the pin key 100 from the keyway through hole 335 and the keyway hole 301 to disengage the connection mount of the needle holder 33 and retracts it into the interior of the barrel 3. That provides a safety mechanism to avoid the second use of the syringe. In addition, the foregoing mechanism is applied in the blood collection system 4, the infusion set needle 5, the arterial venous fistula needle 6 and the butterfly injection needle 7

With reference to FIG. 61, in the present invention of safety syringe, a sticker tape 102 positioned between the inner surface of the barrel 3 and the outer surface of the needle holder 33 for the purpose of engagement is designed in place of the annular check plate 34 formed as a part of inner surface of the barrel 3 adjacent to the connection mount of the needle holder 33. The sticker tape 102 has an appropriate piece of tape extended outside the seam between the inner surface of the barrel 3 and the outer surface of the needle holder 33. After using the syringe for its intended procedure, the healthcare worker only needs to pull out the sticker tape 100 from the seam between the inner surface of the barrel 3 and the outer surface of the needle holder 33 to disengage the connection mount of the needle holder 33 and retracts it into the interior of the barrel 3. That provides a safety mechanism to avoid the second use of the syringe. Additionally, the foregoing mechanism is applied in the blood collection system 4, the infusion set needle 5, the arterial venous fistula needle 6 and the butterfly injection needle 7′

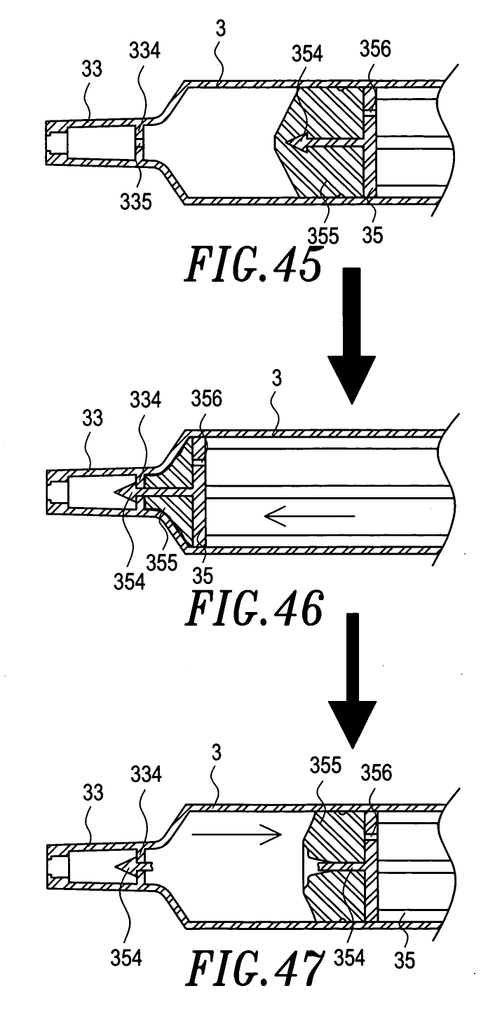

With reference to FIG. 45, FIG. 46, and FIG. 47, the present invention of safety syringe includes:

a barrel 3 having a needle holder 33 in the front portion of the barrel 3, wherein the needle body 1 is positioned, an annular locking plate 334, and a through channel 333 in the annular locking plate 334; and

a piston rod 35, which is positioned into the barrel 3 from rear end, having a spear-like barb 354 surrounding with the rubber wrap 355 and a plurality of leakage holes 356 through the piston rod 35 around the spear-like barb 354.

After the completion of the injection and aspirating, healthcare professionals only need to push the piston rod 35 forward to closely match the rubber wrap 355 with the profile of the rear wall of the annular locking plate 334 and continuously push the piston rod 35 to coerce the spear-like barb 354 to pierce through the rubber wrap 355 and the through channel 333 to lock in the annual locking plate 334. The annular locking plate 334 restrains the spear-like barb from moving backward. In case that the operator brutally pulls the piston rod rearwards, this action fractures the rod. In the meantime, the injection and aspiration mechanism are ruined for the reason that the rubber wrap is pierced through and the air leaks through the leakage holes 356 and the rubber wrap 355. Moreover, another design is proposed to remove the through channel 333 and the annular locking plate 334. After the completion of injection or aspirating, the healthcare professionals can push the piston rod 35 and spear-like barb 354 forward to pierce through the rubber wrap 355. That causes the leakage of air inside the barrel 3 to destruct the mechanism of injection and aspirating of this syringe to avoid the second use of syringe.

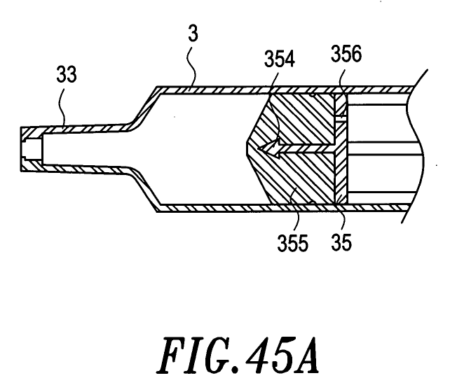

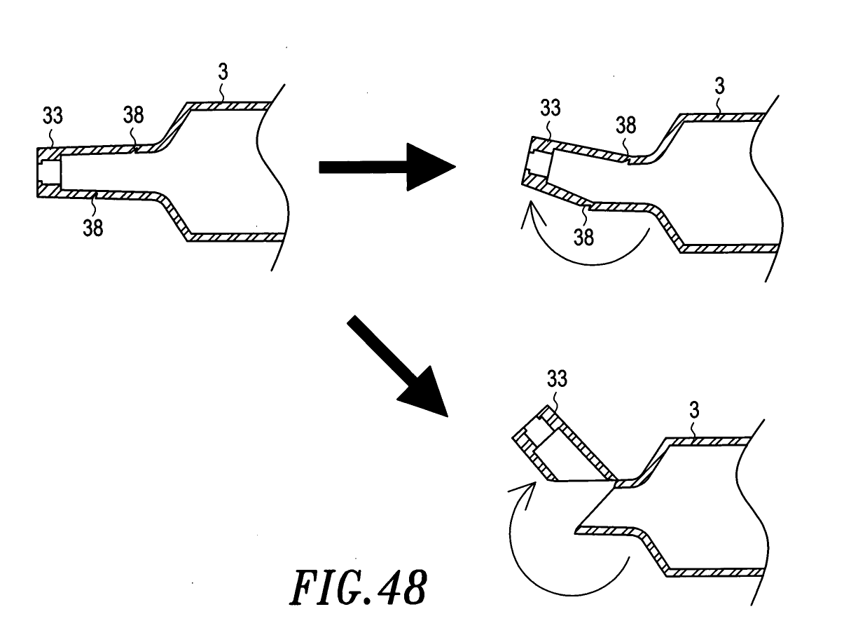

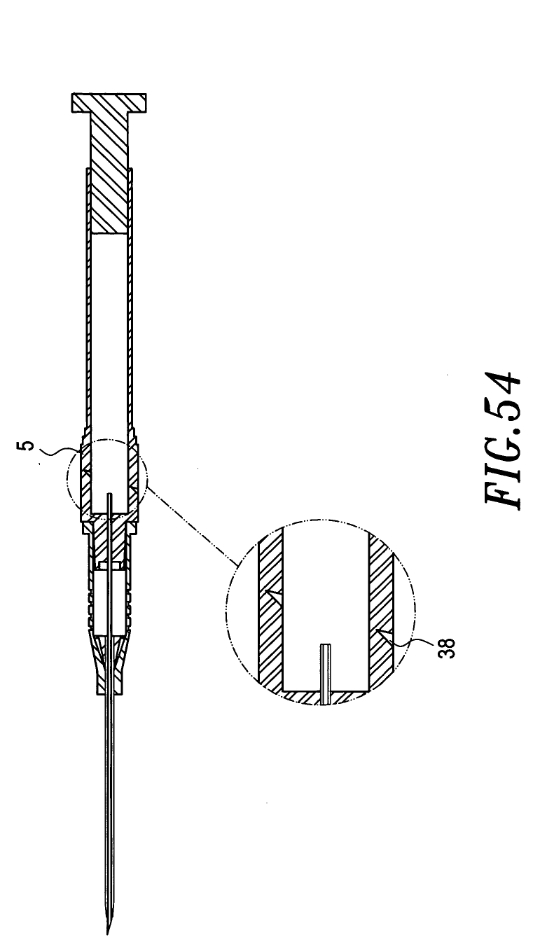

With reference to FIG. 48, in the present invention of safety syringe, two fracture notch 38, 38 is sculptured on the same side of the diametrically opposite walls to replace the annual locking plate 334 positioned inside the needle holder 33, the spear-like barb 354 and the leakage holes 356 in the piston rod 35. After using this safety syringe for its intended purpose, the healthcare workers bend the front portion of the safety syringe the syringe clockwise about the vertex of the fracture notch 38 to fracture it and avoid it for the second use. Additionally, the same structure of the fracture notch 38 is proposed to design in the needle body 1, the blood collection system 4, the infusion set needle 5, the arterial venous fistula needle 6, and the butterfly injection needle 7, as can be referred to FIG. 50, FIG. 52, and FIG. 54.

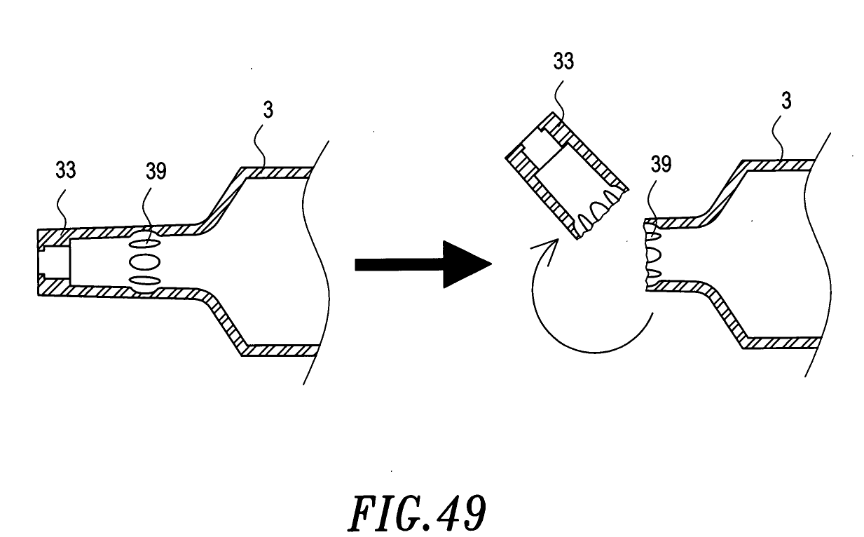

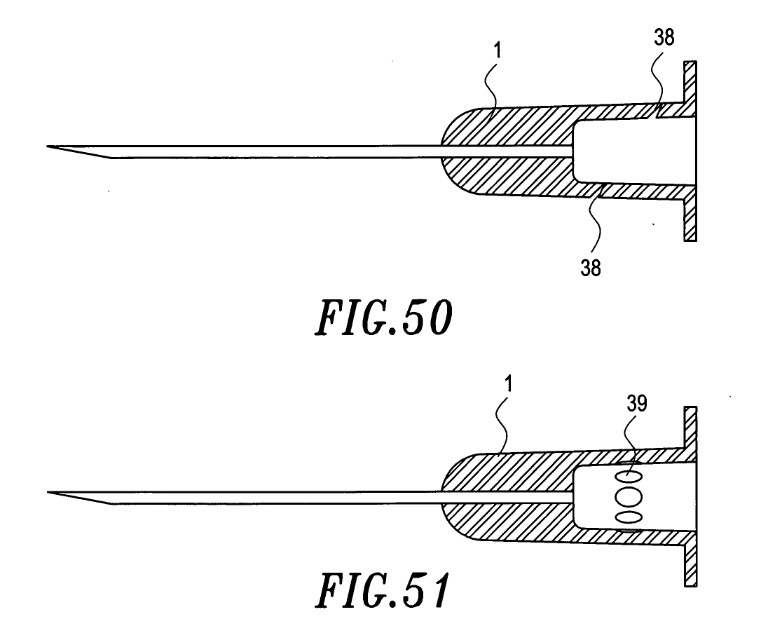

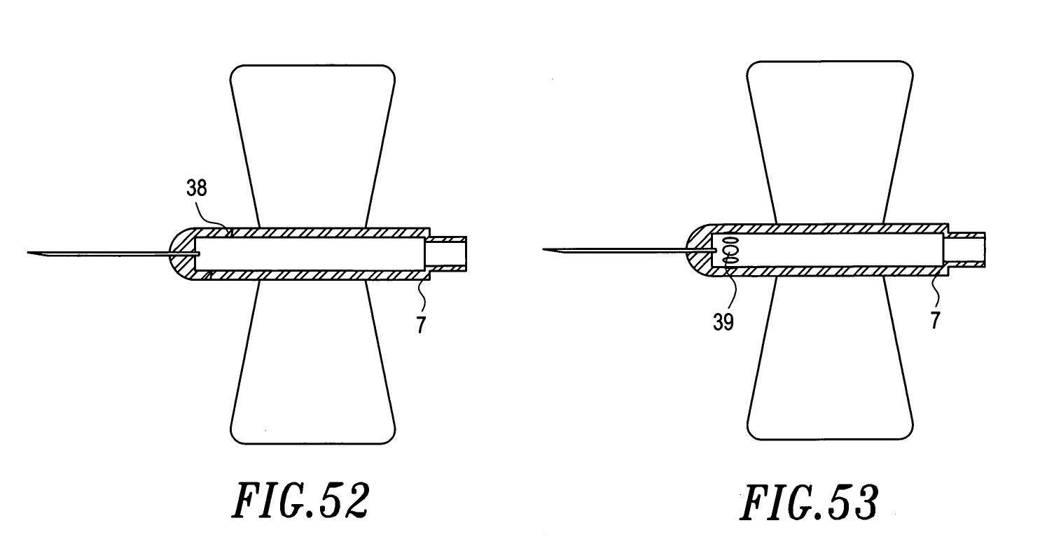

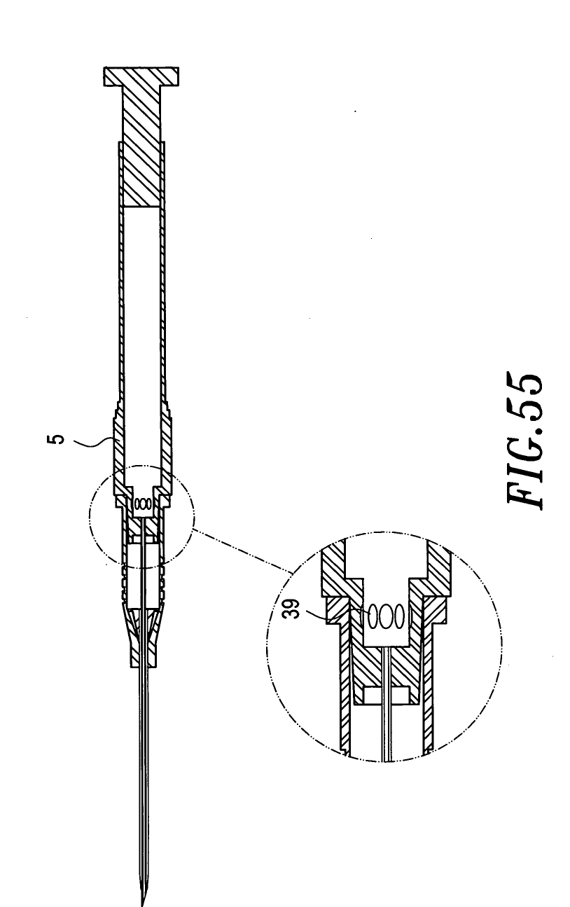

With reference to FIG. 49, in the present invention of safety syringe, a plurality of annular fracture indentions 39 in the interior of the needle holder 33 are used in lieu of the annual locking plate 334 positioned inside the needle holder 33, spear-like barb 354 and the leak holes 356 of the piston rod 35. After the completion of using this syringe for its intended procedure, the healthcare workers bend the front portion of the syringe clockwise about the location of the annular fracture indentions 39 to fracture it and avoid the likelihood for reuse. Additionally, the structure of the annular fracture indentions 39 is proposed to apply in the needle body 1, the blood collection system 4, the infusion set needle 5, the arterial venous fistula needle 6, and the butterfly injection needle 7, as can be referred to FIG. 51, FIG. 53, and FIG. 55.

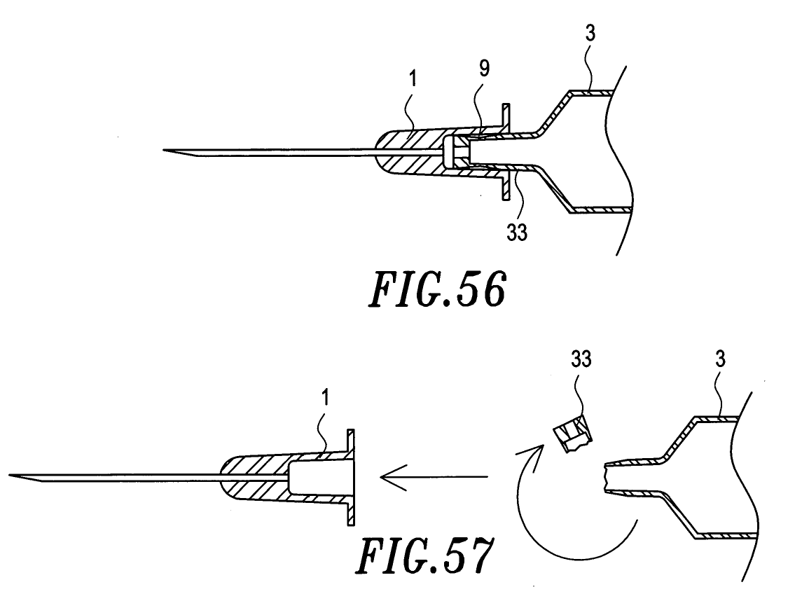

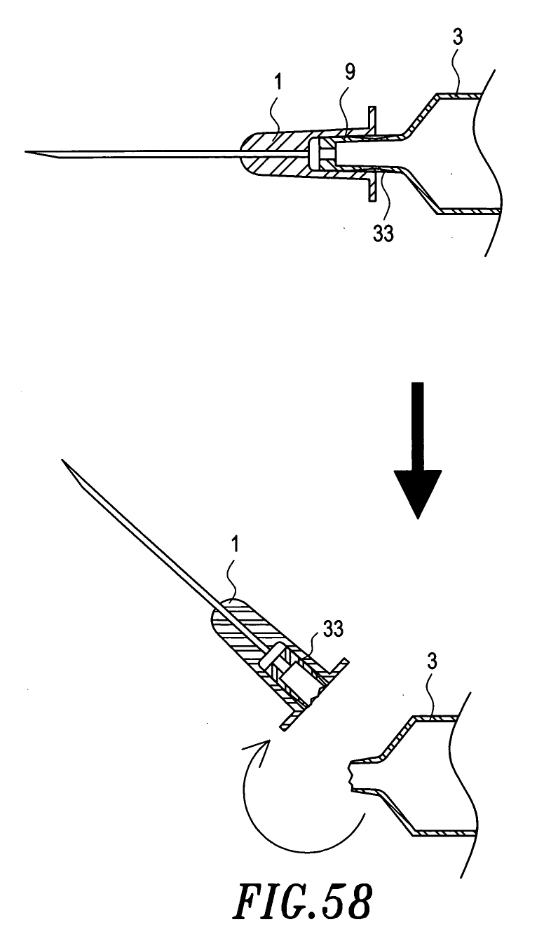

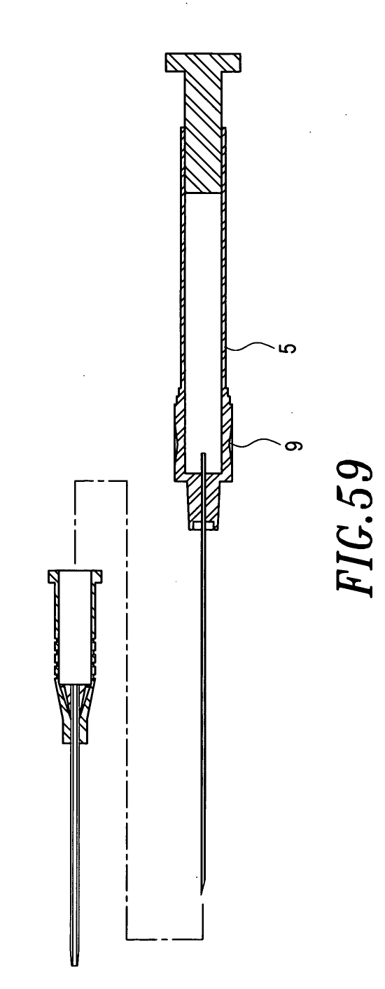

Referring to FIG. 56, FIG. 57, and FIG. 58, in the present invention of safety syringe, a plurality of circumferential U-shape cuts 9 formed in the exterior of the needle holder 33 are proposed instead of the annular locking plate 334 positioned inside the needle holder 33, spear-like barb 354 and the leakage holes 356 of the piston rod 35. After the accomplishment of using this syringe for its intended procedure, the healthcare workers hold the front portion of the safety syringe, bend the syringe clockwise about the location of the circumferential U-shape cut 9 to fracture it and avoid it for reuse. In addition, the structure of the annular fracture indentions 39 is proposed to apply in the outer surface of the needle body 1, the blood collection system 4, the infusion set needle 5, the arterial venous fistula needle 6, and the butterfly injection needle 7.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A safety syringe, more particularly, referring to a safety structure of the barrel comprising the parts of:
a barrel possessing a rubber stopper with a shallow disk indention at the rear end, which is positioned in the interior, the barrel having a structure of annular check rib formed adjacent to the rear end in inner surface of the hollow barrel and a needle holder, the barrel having the needle holder engaging with a needle body infused with the needle cannula; and
an actuator comprising of an electromagnet and a magnet opposite to one another with unlike poles attraction, the actuator inserted from the rear open end into the interior of the barrel, the actuator having the magnet attached on the front surface of the actuator mating with the circular indention on the rear surface of the rubber stopper, the actuator compelling the rubber stopper to move back or forth by means of switching the pole of electromagnet.

2. The safety syringe according to claim 1, wherein the magnet is positioned directly in the rear side of the rubber stopper.

3. The safety syringe according to claim 1, wherein the circular check plate with the through channel in the center is formed as a part of the needle holder and the spear-like barb at the proximal end of the rubber stopper is surrounded with the rubber wrap.

4. The safety syringe according to claim 1, wherein the circular check plate and the through channel are removed in the needle holder.

5. The safety syringe according to claim 1, wherein the needle holder is embedded in the barrel, the locking lumen is formed inside the needle holder, the through channel formed to communicate the locking lumen with the interior of the barrel, an annular check rib formed in abutment to the rear end of the needle holder inside the barrel.

6. A safety syringe, more particularly, refers to a safety structure including:
a needle body having the needle cannula fused at the front end; and
a cap having a shield base, a needle passageway through the shield base, a nozzle-like circumferential check plate formed behind the needle passageway of the shield base, and a retractable elastomeric tube, the cap using the retractable elastomeric tube to connect the rear end of the shield base with the retaining bases of needle holder, which has a connection tab in linkage to a buttoning check plate, the cap shielding the needle cannula inside, the cap having the buttoning check plate to fasten it in the rear or front edge of the needle holder.

7. The safety syringe according to claim 6, wherein the nozzle-like circumferential check plate behind the shield base with the needle passageway is replaced, a button structure for buttoning and locking purpose formed on the outer surface of the shield base and the top surface of the buttoning check plate, an upstanding seat with a needle tip trapping pit at its front end surface formed as a part of the buttoning check plate.

8. The safety syringe according to claim 6, wherein the nozzle-like circumferential check plate behind the shield base and the needle passageway is replaced, a button structure for buttoning and locking purpose formed on the outer surface and the top of a buttoning rectangular space of the shield base where the buttoning check plate is mating with, an upstanding seat with a needle tip trapping pit at its front end surface is formed as a part of the buttoning check plate.

9. The safety syringe according to claim 6, wherein the retractable elastomeric tube is replaced by two lamented leaf springs positioned diametrically opposite to one another in the outer side of needle cannula.

10. The safety syringe according to claim 9, wherein the needle cannula is positioned within a sequence of guided holes on a specifically designed lamented leaf spring.

11. The safety syringe according to claim 6, wherein the shield base is miniaturized and mounted in the outer edge of the needle body, the specifically designed lamented leaf spring compressed to fit the space formed between the shield base and the needle holder, the exterior of the needle cannula covered by a cap.

12. A safety syringe comprising:
a needle body with the fused needle cannula;
a cap with a needle passageway at the front end, a connection tab being the connection to the front cap and the cap, a inner projection bung inside the interior of the front cap, two opposite guided slots in the exterior of the cap, a rear and a front end containing cut and the anchor cut of the guided slot, a plurality of V-shape check ledges between the anchor cut and the rear end containing cut; and
a barrel having two sliding rods with ear tab in both sides of the barrel.

13. The safety syringe according to claim 12, wherein the guided sliding rods and the ear tabs diametrically opposite to one another are formed in the interior of the cap, two opposite guided slots formed in the outer side of the barrel of the syringe, two containing cuts are formed at the end of the guided slot, a plurality of ledges designed between the containing cut and guided slot, a plurality of V-shape check ledges designed between the containing cut and the anchor cut at the rear end of the guided slot.

14. The safety syringe according to claim 12, wherein the guided slot of the cap, the front cap, and the ear tab in the outer side of the barrel are replaced, two spindles formed diametrically opposite to each other in the circumference of the needle holder, a positioning bump which is in shape of a barbed acute angle head in the direction towards the axis structured in one spindle, a bias angle named after the angle between the side of positioning bump and the spindle side near the syringe, an adjusting bump in the shape of obtuse angle head structured in the other spindle, two mounting holes opposite to each other in the rear portion of the cap arranged corresponding to these two spindles, an opening cutout structured between two mounting holes, a nozzle-like circumferential check plate made behind the needle passageway.

15. The safety syringe according to claim 14, wherein the foregoing spindles positioned in the needle body and mounting holes are applied in the blood collection system, the infusion set needle, the arterial venous fistula needle and the butterfly injection needle.

16. The safety syringe according to claim 14, wherein the positioning bump and the adjusting bump are replaced in this design, a rectangular anchor cut connected to the mounting hole, a multiple of the check parts formed between each mounting holes and its rectangular anchor cut, a plurality of angle positioning broached contours used to position the required angle.

17. The safety syringe according to claim 16, wherein the foregoing spindles in the needle body, mounting holes, rectangular anchor cuts, and angle positioning cuts are applied in the blood collection system, the infusion set needle, the arterial venous fistula needle and the butterfly injection needle.

18. The safety syringe according to claim 12, wherein the front cap, the guided slot, and the guided sliding rod are replaced, the upstanding notch connector located between the radically inner side of the cross sliding adjustor and the side surface of the needle body, a plurality of positioning slots arranged longitudinally in line in the outer wall of the cap, a connection groove used as the connection among these positioning slots, the first positioning slot from the rear end of the cap directly connected to the needle containing cavity/housing inside the cap, a nozzle-like circumferential check plate structured behind the needle passageway in the cap, the exterior of the cap 2 and the needle containing cavity designed eccentric.

19. The safety syringe according to claim 18, wherein the upstanding notch connector is removed from the needle body, but are formed on the exterior of a washer which is mounted between the needle body and the barrel.

20. The safety syringe according to claim 18, wherein a positioning hole is in the side surface of the needle body, a positioning adjustor used to replace the upstanding notch connector, an anchor recess with a plurality of annual check plates around the passing opening being at the radially inner end, a positioning cavity being in the middle, and a transition cavity being at the radially outer end, a positioning rod with a positioning ball corresponding to those recess and cavities connected to the radially inner side of the positioning adjustor, a check block formed in the one or both sides of the positioning adjustor.

21. A safety syringe, more particularly, refers to a safety structure of the barrel including:
a barrel with a needle seat inside the front portion of the barrel, which has an annular check rib behind the needle seat;
a piston rod with a linear elastomer groove at the front end; and
a linear elastomer connected around the linear elastomer groove at one end and connected to the rear side of the needle holder at the other end.

22. The safety syringe according to claim 21, wherein the linear elastomer groove is rearranged to the rear side of the needle seat and with the direct connection to the front end of the piston rod.

23. The safety syringe according to claim 21, wherein a linkage rod is connected to a linear elastomer affixed to the front portion of the piston rod and is directly formed at the rear end of the needle holder in order to replace the linear elastomer groove.

24. The safety syringe according to claim 23, wherein the linkage rod is designed through the piston rod in connection with a linear elastomer, which is linking to the front surface of a block at the other end.

25. The safety syringe according to claim 21, wherein the linkage rod is directly connected with the block.

26. The safety syringe according to claim 21, wherein a spear-like barb surrounded by a rubber wrap and a locking lumen formed inside the needle holder, which connects to the interior cavity with the through channel in the front portion of the needle holder is proposed to replace the linear elastomer groove.

27. A safety syringe, more particularly, refers to the safety structure of the syringe, including:
a barrel, which has a needle holder mating with the needle body in the front portion of the barrel and an annular locking plate with a through channel; and
a piston rod, which seats into the barrel from the barrel's proximal end, has a spear-like barb surrounding with the rubber wrap and a plurality of leakage holes around the spear-like barb in the piston rod.

28. The safety syringe according to claim 27, wherein the through channel and the annular locking plate are removed from the needle holder.

29. The safety syringe according to claim 27, wherein two fracture notch is sculptured in the same side of the opposite wall to replace the annular locking plate inside the needle holder, spear-like barb and the leakage holes in the piston rod.

30. The safety syringe according to claim 27, wherein a plurality of annular fracture indentions in the interior of the needle holder are used instead of the annular locking plate inside the needle holder, spear-like barb and the leakage holes of the piston rod.

31. The safety syringe according to claim 29, wherein the same structure of the fracture notch is formed with the needle body, the present invention of safety syringe also applied in the blood collection system, the infusion set needle, the arterial venous fistula needle, and the butterfly injection needle.

32. The safety syringe according to claim 29, wherein the structure of the annular fracture indentions is applied in the outer face of needle body, the blood collection system, the infusion set needle, the arterial venous fistula needle, and the butterfly injection needle.

33. The safety syringe according to claim 27, wherein a plurality of circumferential U-shape cuts in the exterior of the needle holder are proposed instead of the annular locking plate inside the needle holder, spear-like barb and the leakage holes of the piston rod.

34. The safety syringe according to claim 33, wherein the structure of the annular fracture indentions is applied in the outer surface of the needle body, the blood collection system, the infusion set needle, the arterial venous fistula needle, and the butterfly injection needle.

35. The safety syringe according to claim 21, wherein a keyway through hole formed in the front portion of the side wall of the barrel, a keyway hole formed in the position of the connection mount of the needle holder corresponding to the keyway through hole, and a pin key inserted through the keyway through hole and keyway hole to engage the connection mount of the needle holder with the front portion of the barrel are proposed instead of the annular check plate formed as a part of inner surface of the barrel adjacent to the connection mount of the needle holder.

36. The safety syringe according to claim 35, wherein the keyway through hole, the keyway hole and the pin key are applied in the blood collection system, the infusion set needle, the arterial venous fistula needle, and the butterfly injection needle.

37. The safety syringe according to claim 21, wherein a sticker tape positioned between the inner surface of the barrel and the outer surface of the needle holder for the purpose of engagement is designed in place of the annular check plate formed as a part of inner surface of the barrel adjacent to the connection mount of the needle holder, the sticker tape having an appropriate piece of tape extended outside the seam between the inner surface of the barrel and the outer surface of the needle holder.

38. The safety syringe according to claim 37, wherein the sticker tape is implemented in the blood collection system, the infusion set needle, the arterial venous fistula needle, and the butterfly injection needle.
